(19) United States
(12) Patent Application Publication
(10) Pub. No.: US 2024/0360454 A1
(43) Pub. Date: Oct. 31, 2024

(54) RENAL INJURY BIOMARKERS AS BIOMARKERS FOR ACUTE HEPATIC PORPHYRIA (AHP)

(71) Applicant: Alnylam Pharmaceuticals, Inc., Cambridge, MA (US)

(72) Inventors: Simina Ticau, Braintree, MA (US); Anna Borodovsky, Melrose, MA (US)

(21) Appl. No.: 18/623,281

(22) Filed: Apr. 1, 2024

Related U.S. Application Data
(63) Continuation of application No. PCT/US2022/077511, filed on Oct. 4, 2022.
(60) Provisional application No. 63/252,919, filed on Oct. 6, 2021.

(51) Int. Cl. C12N 15/113; A61K 31/555; A61K 31/7004; A61P 1/16; G01N 33/68

(52) U.S. Cl. CPC C12N 15/1137; A61K 31/555; A61K 31/7004; A61P 1/16; G01N 33/6893; C12N 2310/11; C12N 2310/14; G01N 2800/08; G01N 2800/60

(57) ABSTRACT

The disclosure provides biomarkers for diagnosis and monitoring of acute hepatic porphyria (AHP). The disclosure further provides methods for selection of agents for treatment of AHP using the biomarkers. The disclosure further provides kits for practicing the methods provided herein.

### RELATED APPLICATIONS

This application is a 35 § U.S.C. 111(a) continuation application which claims the benefit of priority to PCT/US2022/077511, filed on Oct. 4, 2022, which, in turn, claims the benefit of priority to U.S. Provisional Application No. 63/252,919, filed on Oct. 6, 2021. The entire contents of the foregoing applications are incorporated herein by reference.

### SEQUENCE LISTING

The instant application contains a Sequence Listing XML which has been submitted electronically in XML format and is hereby incorporated by reference in its entirety. Said XML copy, created on Mar. 28, 2024, is named 121301-18802_SL.xml and is 75,394 bytes in size.

### FIELD OF THE DISCLOSURE

The disclosure relates to biomarkers for acute hepatic porphyria (AHP). More specifically, the disclosure relates to renal injury biomarkers as biomarkers for AHP.

### BACKGROUND

Acute hepatic porphyria (AHP) is a group of rare genetic diseases caused by defects in enzymes in the heme biosynthesis pathway. AHP has four subtypes: acute intermittent porphyria (AIP), variegate porphyria (VP), hereditary coproporphyria (HCP), and ALAD-deficiency porphyria (ADP). The most common subtype is AIP.

In patients with AHP, accumulation of heme pathway intermediates, delta-aminolevulinic acid (ALA) and porphobilinogen (PBG), lead to acute attacks and long-term complications including hypertension and chronic kidney disease which is present in 30-60% of patients with biochemically active AIP.

### SUMMARY

In accordance with one aspect, there is provided a method of treating a human subject having acute hepatic porphyria (AHP). The method comprises, for example, responsive to a determination of an elevated level of a biomarker (e.g., a renal injury biomarker) in a subject relative to a reference level, administering to the subject a therapeutic agent that reduces expression of 5′-aminolevulinic acid synthase 1 (ALAS1), thereby treating the subject.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter (CHE). In some embodiments, the subject has recurring acute attacks. In some embodiments, the subject has not been diagnosed with AHP. In some embodiments, the subject has not been diagnosed as having a porphyria. In some embodiments, the subject does not meet the diagnostic criteria for AHP. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

In accordance with another aspect, there is provided a method of treating a human subject having, or at risk of having, AHP. The method comprises, for example, obtaining or having obtained a biological sample from the subject, performing or having performed an assay to determine the level of a biomarker (e.g., a renal injury biomarker) in the biological sample, and, if the subject has an elevated level of the biomarker (e.g., renal injury biomarker) relative to a reference value, administering to the subject a therapeutic agent that reduces expression of ALAS1.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter. In some embodiments, the subject has recurring acute attacks. In some embodiments, the subject has not been diagnosed with AHP. In some embodiments, the subject has not been diagnosed as having a porphyria. In some embodiments, the subject does not meet the diagnostic criteria for AHP. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

In accordance with another aspect, there is provided a method of treating a human subject having, or at risk of having, AHP. The method can comprise providing a therapeutic agent that reduces the expression of ALAS1, detecting an elevated level of a biomarker (e.g., a renal injury biomarker) in the subject relative to a reference level, and administering the therapeutic agent to the subject if the level of the biomarker (e.g., renal injury biomarker) is greater than the reference level.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter. In some embodiments, the subject has recurring acute attacks. In some embodiments, the subject has not been diagnosed with AHP. In some embodiments, the subject has not been diagnosed as having a porphyria. In some embodiments, the subject does not meet the diagnostic criteria for AHP. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

In accordance with another aspect, there is provided a therapeutic agent that reduces expression of ALAS1 for use in treating a human subject with AHP wherein the subject has an elevated level of a biomarker (e.g., a renal injury biomarker) as compared to a reference level.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter. In some embodiments, the subject has recurring acute attacks. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

In accordance with another aspect, there is provided a therapeutic agent that reduces expression ALAS1 for use in a method of treatment of a human subject with AHP, wherein the method comprises the step of determining if a patient has an elevated level of a biomarker (e.g., a renal injury biomarker) as compared to a reference level of the renal injury biomarker.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter. In some embodiments, the subject has recurring acute attacks. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

In accordance with another aspect, there is provided an in vitro method of diagnosing AHP in a subject. The method can comprise, for example, (a) determining the level of a biomarker (e.g., a renal injury biomarker) in a sample from the subject; (b) comparing the level of the biomarker (e.g., renal injury biomarker) determined in step (a) to a reference level of the biomarker (e.g., renal injury biomarker); and (c) assessing whether the subject suffers from AHP, wherein an increase in the level of the biomarker (e.g., renal injury biomarker) determined in step (a) as compared to the reference level of the biomarker (e.g., renal injury biomarker) is indicative of the subject suffering from AHP.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter. In some embodiments, the subject has recurring acute attacks. In some embodiments, the subject has not been diagnosed with AHP. In some embodiments, the subject has not been diagnosed as having a porphyria. In some embodiments, the subject does not meet the diagnostic criteria for AHP. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

In accordance with another aspect, there is provided a therapeutic agent that reduces expression of ALAS1 for use in the treatment of AHP in a subject that has been identified as suffering from AHP using a method as defined herein.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter. In some embodiments, the subject has recurring acute attacks. In some embodiments, the subject has not been diagnosed with AHP. In some embodiments, the subject has not been diagnosed as having a porphyria. In some embodiments, the subject does not meet the diagnostic criteria for AHP. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

In accordance with another aspect, there is provided a therapeutic agent that reduces expression of ALAS1 for use in a method of treating AHP. The method can comprise, for example, (a) determining the level of a biomarker (e.g., a renal injury biomarker) in a sample from the subject; (b) comparing the level of the biomarker (e.g., renal injury biomarker) determined in step (a) to a reference level of the biomarker (e.g., renal injury biomarker); (c) assessing whether the subject suffers from AHP, wherein an increase in the level of the biomarker (e.g., renal injury biomarker) determined in step (a) as compared to the reference level of the biomarker (e.g., renal injury biomarker) is indicative of the subject suffering from AHP; and (d) administering the therapeutic agent that reduces expression of ALAS1 to a subject that has been identified in step (c) as suffering from AHP.

In some embodiments, the biomarker is chosen from one or more (e.g., 2, 3, 4, 5, or all) of KIM1, APLP1, MMP7, NGAL, CST3, or CHI3L1.

In some embodiments, the biomarker is a renal injury biomarker. In some embodiments, the renal injury biomarker is chosen from one or more (e.g., 2, 3, 4, or all) of KIM1, MMP7, NGAL, CST3, or CHI3L1.

In some embodiments, the therapeutic agent that reduces the expression of ALAS1 is a nucleic acid therapeutic. In some embodiments, the nucleic acid therapeutic is an RNAi agent or an antisense oligonucleotide. In some embodiments, the nucleic acid therapeutic is an RNAi agent described herein. In some embodiments, the nucleic acid therapeutic is givosiran.

In some embodiments, the subject is a chronic high excreter. In some embodiments, the subject has recurring acute attacks. In some embodiments, the subject has not been diagnosed with AHP. In some embodiments, the subject has not been diagnosed as having a porphyria. In some embodiments, the subject does not meet the diagnostic criteria for AHP. In some embodiments, the subject has an elevated level of ALA and/or PBG. In some embodiments, the subject has a mutation associated with AHP. In some embodiments, the subject does not have a mutation associated with AHP. In some embodiments, the subject has a history of renal impairment. In some embodiments, the subject has been diagnosed with a renal injury. In some embodiments, the subject has a reduced estimated glomerular filtration rate (eGFR). In some embodiments, the subject is further suffering from one or more symptoms associated with AHP.

In some embodiments, the reference level is a healthy control level or an earlier level in the same subject. In some embodiments, the level of the biomarker (e.g., renal injury biomarker) is increased by at least 2-fold (e.g., at least 3, 4, 5, 6, 7, 8, 9, or 10-fold), compared to the reference level of the biomarker (e.g., renal injury biomarker). In some embodiments, the level is determined in a sample chosen from blood, plasma, serum, urine, or stool, from the subject.

In some embodiments, the subject is being treated with a second therapeutic agent. In some embodiments, the second therapeutic agent comprises a heme product (e.g., hemin, heme arginate, or heme albumin), glucose (e.g. IV glucose), dextrose, or combinations thereof. In some embodiments, the method further comprises discontinuation of treatment with the second therapeutic agent when the subject has the elevated level of the biomarker (e.g., renal injury biomarker).

### BRIEF DESCRIPTION OF THE DRAWINGS

FIGS. 1A-1B includes graphs showing the difference in protein expression of plasma proteins between patients with AHP in two cohorts and healthy controls.

FIGS. 2A-2C includes graphs showing kidney injury molecule-1 (KIM1) levels in the different populations studied, KIM1 levels in patients with a history of renal impairment, and KIM1 levels plotted against estimated glomerular filtration rate (eGFR).

FIGS. 3A-3C includes graphs showing matrix metalloproteinase-7 (MMP7) levels in the different populations studied, MMP7 levels in patients with a history of renal impairment, and MMP7 levels plotted against eGFR.

FIGS. 4A-4C includes graphs showing neutrophil gelatinase-associated lipocalin (NGAL) levels in the different populations studied, NGAL levels in patients with a history of renal impairment, and NGAL levels plotted against eGFR.

FIGS. 5A-5C includes graphs showing cystatin 3 (CST3) levels in the different populations studied, CST3 levels in patients with a history of renal impairment, and CST3 levels plotted against eGFR.

FIGS. 6A-6C includes graphs showing chitinase-3-like protein 1 (CHI3L1) levels in the different populations studied, CHI3L1 levels in patients with a history of renal impairment, and CHI3L1 levels plotted against eGFR.

### DETAILED DESCRIPTION

The inherited porphyrias are a family of disorders resulting from the deficient activity of specific enzymes in the heme biosynthetic pathway, also referred to herein as the porphyrin pathway. Deficiency in the enzymes of the porphyrin pathway leads to insufficient heme production and to an accumulation of porphyrin precursors and porphyrins, which are toxic to tissue in high concentrations.

Of the inherited porphyrias, acute intermittent porphyria (AIP, e.g., autosomal dominant AIP), variegate porphyria (VP, e.g., autosomal dominant VP), hereditary coproporphyria (coproporphyria or HCP, e.g., autosomal dominant HCP), and 5′ aminolevulinic acid (also known as δ-aminolevulinic acid or ALA) dehydratase deficiency porphyria (ADP, e.g., autosomal recessive ADP) are classified as acute hepatic porphyrias and are manifested by acute neurological attacks that can be life threatening. The acute attacks are characterized by autonomic, peripheral, and central nervous symptoms, including severe abdominal pain, hypertension, tachycardias, constipation, motor weakness, paralysis, and seizures. If not treated properly, quadriplegia, respiratory impairment, and death may ensue. Various factors, including cytochrome P450-inducing drugs, dieting, and hormonal changes can precipitate acute attacks by increasing the activity of hepatic 5′-aminolevulinic acid synthase 1 (ALAS1), the first and rate-limiting enzyme of the heme biosynthetic pathway. In the acute porphyrias, e.g., AIP, VP, HCP and ADP, the respective enzyme deficiencies result in hepatic production and accumulation of one or more substances (e.g., porphyrins and/or porphyrin precursors, e.g., ALA and/or PBG) that can be neurotoxic and can result in the occurrence of acute attacks. See, e.g., Balwani, M and Desnick, R. J., Blood, 120:4496-4504, 2012.

AIP, also referred to as porphobilinogen deaminase (PBGD) deficiency, or hydroxymethylbilane synthase (HMBS) deficiency, is the most common of the acute hepatic prophyrias. The prevalence of AIP is estimated to be 5-10 in 100,000, with about 5-10% of patients being symptomatic. AIP is an autosomal dominant disorder caused by mutations in the HMBS gene that result in reduced, e.g., half-normal activity of the enzyme.

Currently, diagnosis of the disease is challenging. Diagnosis of porphyria can involve assessment of family history, assessment of porphyrin precursor levels in urine, blood, or stool, and/or assessment of enzyme activity and DNA mutation analysis. The differential diagnosis of porphyrias may involve determining the type of porphyria by measuring individual levels of porphyrins or porphyrin precursors (e.g., ALA, PBG) in the urine, feces, and/or plasma (e.g., by chromatography and fluorometry). However, samples must generally be obtained during an attack. The diagnosis of AIP can be confirmed by establishing that erythrocyte PBG deaminase activity is at 50% or less of the normal level. DNA testing for mutations may be another method of diagnosing AIP.

AHP may present in the form of recurring acute attacks or chronic high excreters (CHE). CHE are a group of AHP patients that carry a genetic mutation and have elevated levels of ALA and PBG but are not experiencing acute attacks. Diagnosis of CHE can be more challenging. DNA testing for mutations may be required for patients and at-risk family members. Thus, the diagnosis of AHP, in particular, AIP typically requires confirmation by DNA testing and identification of a specific causative gene mutation (e.g., an HMBS mutation).

Treatment of acute attacks typically requires hospitalization for control and treatment of acute symptoms, including, e.g., abdominal pain, seizures, dehydration/hyponatremia, nausea/vomiting, tachycardia/hypertension, urinary retention/ileus. For example, abdominal pain may be treated, e.g., with narcotic analgesics, seizures may be treated with seizure precautions and possibly medications (although many anti-seizure medications are contraindicated), nausea/vomiting may be treated, e.g., with phenothiazines, and tachycardia/hypertension may be treated, e.g., with beta blockers. Treatment may include withdrawal of unsafe medications, monitoring of respiratory function, as well as muscle strength and neurological status. Mild attacks (e.g., those with no paresis or hyponatremia) may be treated with at least 300 g intravenous 10% glucose per day, although increasingly hemin is provided immediately. Severe attacks are typically treated as soon as possible with intravenous hemin (3-4 mg/kg daily for 4-14 days) and with IV glucose while waiting for the IV hemin to take effect. Typically, attacks are treated with IV hemin for 4 days and with IV glucose while waiting for administration of the IV hemin. Within 3-4 days following the start of hemin administration, there is usually clinical improvement accompanying by lowering of ALA and PBG levels.

Hemin (PANHEMATIN® or hemin for injection, previously known as hematin) is one current therapy for acute neurologic attacks. PANHEMATIN® is hemin derived from processed red blood cells (PRBCs) and is Protoporphyrin IX containing a ferric iron ion (Heme B) with a chloride ligand. Heme acts to limit the hepatic and/or marrow synthesis of porphyrin. The exact mechanism by which hemin produces symptomatic improvement in patients with acute episodes of the hepatic porphyrias has not been elucidated; however, its action is likely due to the (feedback) inhibition of δ-aminolevulinic acid (ALA) synthase, the enzyme which limits the rate of the porphyrin/heme biosynthetic pathway. It is believed that PANHEMATIN® provides exogenous heme for the negative feedback inhibition of ALAS1, and thereby, decreases production of ALA and PBG. While patients generally respond well, its effect is relatively slow to normalize urinary ALA and PBG concentrations towards normal levels. As the intravenous hemin is rapidly metabolized, three to four infusions are usually necessary to effectively treat or prevent an acute attack.

Givosiran (GIVLAARI®) is an AHP therapeutic agent that targets and causes degradation of ALAS1 mRNA, reducing the production of neurotoxic intermediates ALA and PBG. Clinical trials for GIVLAARI® have demonstrated rapid and sustained reductions in ALA and PBG.

Delayed administration of a therapeutic agent or continued exposure to precipitating factors can lead to more serious complications, including motor neuropathy and accompanying symptoms (e.g., weakness, paresis). Respiratory failure and paralysis can occur in severe cases. Recovery from neurological symptoms can take much longer to resolve. Accordingly, rapid and accurate detection and diagnosis can reduce the incidence of serious complications in patients with AHP.

To gain additional insights into AHP and identify changes in the proteome associated with AHP, a plasma proteomics analysis in AHP patients, including patients that experience recurring acute attacks and chronic high excreters (CHE), was performed to understand the proteome in more detail. The study identified plasma biomarkers associated with AHP that can be used to provide a minimally invasive measure that can facilitate earlier patient diagnosis and improved therapeutic intervention.

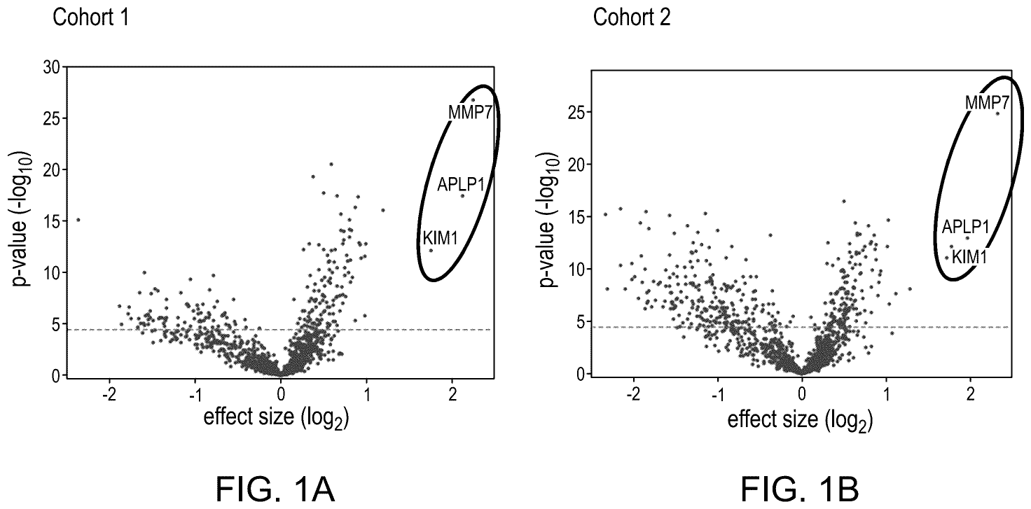

In the analysis, 1196 unique proteins were measured across samples from multiple cohorts. It was found that the plasma levels of 212 proteins were significantly different between healthy controls and patients with AHP. The most significant proteins include amyloid-like protein 1 (APLP1), kidney injury molecule-1 (KIM1) and matrix metalloproteinase-7 (MMP7) (FIGS. 1A-1B). Two of the most significant proteins, KIM1 and MP7, are biomarkers for renal injury in some contexts. At baseline, the AHP patients have significantly higher levels of APLP1, KIM1, and MMP7 compared to healthy controls. Other renal injury biomarkers were explored based on the plasma proteomics analysis and characterization of plasma levels in renal injury. Three additional renal injury biomarkers, neutrophil gelatinase-associated lipocalin (NGAL), cystatin 3 (CST3), and chitinase-3-like protein 1 (CHI3L1) showed significant elevations in patients with AHP.

One of the challenges in treating AHP is the delay in obtaining a proper diagnosis. Now, as there are effective treatments for AHP, there is a greater urgency to promptly identify and treat subjects with AHP. Although DNA may be conveniently sampled and analyzed by health care professionals, the time and skill required to perform an assessment for AHP causative gene mutations is not within the scope of the general practitioner in routine care. The disclosure provides protein biomarkers, including renal injury biomarkers, to evaluate a subject having, or at risk of having, AHP, e.g., a subject having an elevated level of ALA and/or PBG, or a subject with a mutation in an AHP associated gene. Early detection of an elevated biomarkers (e.g., renal injury biomarker) level can be used to prompt initiation of treatment of a subject with an agent that reduces the expression of ALAS1 to treat AHP before the development of overt symptoms.

The disclosure further provides for a method to select a therapeutic agent for treatment of a subject with AHP based on the level of biomarkers (e.g., renal injury biomarkers), including, but not limited to, APLP1, KIM1, MMP7, NGAL, CST3, and/or CHI3L1. Agents that reduce the expression of ALAS1 have been demonstrated in in pivotal trials to be effective in the treatment of AHP, including subjects that experience recurring acute attacks and CHE.

The disclosure further provides diagnostic kits for detection of biomarkers (e.g., renal injury biomarkers), including, but not limited to, APLP1, KIM1, MMP7, NGAL, CST3, and/or CHI3L1, for use in the methods of disclosed herein.

### Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means±10%. In certain embodiments, about means±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range.

As used herein, “or” is understood as “and/or” unless context dictates otherwise.

As used herein, methods of detection can include determination that the amount of analyte present is below the level of detection of the method.

As used herein, an “AHP therapeutic agent” is understood as a therapeutic agent that reduces one or more symptom of AHP. The AHP therapeutic agent may be, for example, a therapeutic agent that reduces expression of ALAS1, or a therapeutic agent that stabilizes ALAS1. In some embodiments, the AHP therapeutic agent prevents the production and accumulation of ALA and/or PBG.

As used herein, a “therapeutic agent that reduces expression of ALAS1” and the like as used herein is understood as a therapeutic agent that reduces levels of ALAS1 RNA, ALAS1 protein, or both of ALAS1 RNA and ALAS1 protein. In some embodiments, the therapeutic agent that reduces expression of ALAS1 is a therapeutic agent that promotes the degradation of an mRNA encoding ALAS1 or inhibits the translation of an mRNA encoding ALAS1. Such agents include, but are not limited to, nucleic acid therapeutics, e.g., RNAi interference agents and antisense oligonucleotide agents. Such agents can typically inhibit expression of both wild type and mutant ALAS1. The amount of ALAS1 in the subject is reduced, thereby reducing the production and accumulation of ALA and/or PBG. In some embodiments, the agent is an iRNA.

As used herein, “renal injury biomarker” or “kidney injury biomarker” is understood as at least a fragment of the sequence of a human polypeptide which has been previously associated with a renal or kidney injury. For example, the injury may be a chronic renal or kidney injury or an acute renal or kidney injury.

As used herein, “kidney injury molecule-1” or “KIM1” is understood as at least a fragment of the sequence of human KIM1 polypeptide, for example, Accession Nos. NP_036338.2 (SEQ ID NO: 1), NP_001166864.1 (SEQ ID NO: 2), or NP_001295085.1 (SEQ ID NO: 3). In some embodiments, KIM1 can be specifically identified by any clinically acceptable diagnostic method, e.g., antibody-based identification method, e.g., ELISA assay or immunoblotting; chromatography method; or single-molecule array (SIMOA).

As used herein, “matrix metalloprotease-7” or “MMP7” is understood as at least a fragment of the sequence of human MMP7 polypeptide, for example, Accession No. NP_002414.1 (SEQ ID NO: 4). In some embodiments, MMP7 can be specifically identified by any clinically acceptable diagnostic method, e.g., antibody-based identification method, e.g., ELISA assay or immunoblotting; chromatography method; or single-molecule array (SIMOA).

As used herein, “neutrophil gelatinase-associated lipocalin” or “NGAL” is understood as at least a fragment of the sequence of human NGAL polypeptide, for example, Accession No. NP_005555.2 (SEQ ID NO: 5). In some embodiments, NGAL can be specifically identified by any clinically acceptable diagnostic method, e.g., antibody-based identification method, e.g., ELISA assay or immunoblotting; chromatography method; or single-molecule array (SIMOA).

As used herein, “cystatin 3” or “CST3” is understood as at least a fragment of the sequence of human CST3 polypeptide, for example, Accession Nos. NP_000090.1 (SEQ ID NO: 6) or NP_001275543.1 (SEQ ID NO: 7). In some embodiments, CST3 can be specifically identified by any clinically acceptable diagnostic method, e.g., antibody-based identification method, e.g., ELISA assay or immunoblotting; chromatography method; or single-molecule array (SIMOA).

As used herein, “chitinase-3-like protein 1” or “CHI3L1” is understood as at least a fragment of the sequence of human CHI3L1 polypeptide, for example, Accession No. NP_001267.2 (SEQ ID NO: 8). In some embodiments, CHI3L1 can be specifically identified by any clinically acceptable diagnostic method, e.g., antibody-based identification method, e.g., ELISA assay or immunoblotting; chromatography method; or single-molecule array (SIMOA).

As used herein, “amyloid-like protein 1” or “APLP1” is understood as at least a fragment of the sequence of human APLP1 polypeptide, for example, Accession No. NP_005157.1 (SEQ ID NO: 9) or NP_001019978.1 (SEQ ID NO: 10). In some embodiments, APLP1 can be specifically identified by any clinically acceptable diagnostic method, e.g., antibody-based identification method, e.g., ELISA assay or immunoblotting; chromatography method; or single-molecule array (SIMOA).

As used herein, a “reference level” is understood as a predetermined level to which a level obtained from an assay, e.g., a biomarker level, e.g., a protein biomarker level, is compared. In certain embodiments, a reference level can be a control level determined for a healthy population, e.g., a population that does not have a disease or condition associated with a changed level of the biomarker and does not have a predisposition, e.g., genetic predisposition, to a disease or condition associated with a changed level of the biomarker. In certain embodiments, the population should be matched for certain criteria, e.g., age, gender. In certain embodiments, the reference level of the biomarker is a level from the same subject at an earlier time, e.g., before the development of symptomatic disease or before the start of treatment. Typically, samples are obtained from the subject at clinically relevant intervals, e.g., at intervals sufficiently separated in time that a change in the biomarker could be observed, e.g., at least three-month interval, at least a six-month interval, or at least a nine-month interval. When more than two samples are obtained from a subject over time, it is understood that any of the prior samples can act as a reference level.

As used herein, a “change as compared to a reference level” and the like is understood as a statistically or clinically significant change in the biomarker level, e.g., the change in the protein biomarker level, as compared to the reference level, is greater than the typical standard deviation of the assay method. Moreover, the change should be clinically relevant. The change as compared to a reference level can be determined as a percent change. For example, if a reference level is 100 pg/ml for biomarker X, and the level of biomarker X in the subject is 150 pg/ml, the level is increased by 50% calculated by ((150 pg/ml-100 pg/ml)/100 pg/ml)×100%=50%. If the level of biomarker X in the subject is 300 pg/ml, the level is increased by 300%. If the level of biomarker X in the subject is 50 pg/ml, the level is decreased by 50%. In certain embodiments, the change as compared to a reference level is increased by at least 50%. In certain embodiments, the change as compared to a reference level is increased by at least 100%, at least 200%, or at least 300%. In certain embodiments, the change as compared to a reference sample is decreased by at least 25%. In certain embodiments, the change as compared to a reference sample is decreased by at least 50%.

A “biological sample from a subject” or a “sample from a subject” as used herein, includes one or more fluids, cells, or tissues isolated from a subject. Examples of biological fluids include blood, serum, serosal fluids, plasma, cerebrospinal fluid, ocular fluids, lymph, urine, stool, saliva, and the like. Tissue samples may include samples from tissues, organs, or localized regions. For example, samples may be derived from particular organs, parts of organs, or fluids or cells within those organs. In certain embodiments, samples may be liver tissue or be derived from the liver. In some embodiments, a “biological sample from a subject” can refer to blood or blood derived serum or plasma from the subject. In some embodiments, the fluid is substantially free of cells, e.g., is free of cells.

As used herein, the term “administering a therapeutic agent” is understood as providing a therapeutic agent to a subject. In embodiments, the therapeutic agent is provided at an appropriate dosage and by a route of administration for the agent as provided, for example, by the label of the therapeutic agent.

As used herein, “ALAS1” (also known as ALAS-1; δ-aminolevulinate synthase 1; δ-ALA synthase 1; 5′-aminolevulinic acid synthase 1; ALAS-H; ALASH; ALAS-N; ALAS3; EC2.3.1.37; 5-aminolevulinate synthase, nonspecific, mitochondrial; ALAS; MIG4; OTTHUMP00000212619; OTTHUMP00000212620; OTTHUMP00000212621; OTTHUMP00000212622; migration-inducing protein 4; EC 2.3.1) refers to a nuclear-encoded mitochondrial enzyme that is the first and typically rate-limiting enzyme in the mammalian heme biosynthetic pathway. ALAS1 catalyzes the condensation of glycine with succinyl-CoA to form δ-aminolevulinic acid (ALA). The human ALAS1 gene is expressed ubiquitously, is found on chromosome 3p21.1 and typically encodes a sequence of 640 amino acids. In contrast, the ALAS-2 gene, which encodes an isozyme, is expressed only in erythrocytes, is found on chromosome Xp11.21, and typically encodes a sequence of 550 amino acids.

As used herein an “ALAS1 protein” means any protein variant of ALAS1 from any species (e.g., human, mouse, non-human primate), as well as any mutants and fragments thereof that retain an ALAS1 activity. Similarly, an “ALAS1 transcript” refers to any transcript variant of ALAS1, from any species (e.g., human, mouse, non-human primate). A sequence of a human ALAS1 mRNA transcript can be found, for example, at NM_000688.4 (SEQ ID NO: 11). The level of the mature encoded ALAS1 protein is regulated by heme: high levels of heme down-regulate the mature enzyme in mitochondria while low heme levels up-regulate. Multiple alternatively spliced variants, encoding the same protein, have been identified.

As used herein, the term “iRNA,” “RNAi”, “iRNA agent,” or “RNAi agent” refers to an agent that contains RNA as that term is defined herein, and which mediates the targeted cleavage of an RNA transcript, e.g., via an RNA-induced silencing complex (RISC) pathway. In one embodiment, an iRNA as described herein effects inhibition of ALAS1 expression. Inhibition of ALAS1 expression may be assessed based on a reduction in the level of ALAS1 mRNA or a reduction in the level of the ALAS1 protein. As used herein, “target sequence” refers to a contiguous portion of the nucleotide sequence of an mRNA molecule formed during the transcription of an ALAS1 gene, including mRNA that is a product of RNA processing of a primary transcription product. The target portion of the sequence will be at least long enough to serve as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides, 20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, a “nucleic acid therapeutic agent” is understood as a therapeutic agent comprising a sufficient length of nucleotides to specifically hybridize to a target sequence in a target nucleic acid in a cell such that the hybridization reduces levels of a protein encoded by the target nucleic acid, e.g., by inhibiting translation or promoting sequence specific degradation of the target nucleic acid. Exemplary nucleic acid therapeutic agents include RNAi agents and antisense oligonucleotide agents.

The terms “antisense polynucleotide agent”, “antisense oligonucleotide”, “antisense compound”, and “antisense agent” as used interchangeably herein, refer to an agent comprising a single-stranded oligonucleotide that specifically binds to the target nucleic acid molecules via hydrogen bonding (e.g., Watson-Crick, Hoogsteen, or reversed Hoogsteen hydrogen bonding) and inhibits the expression of the targeted nucleic acid by an antisense mechanism of action, e.g., by RNase H. In some embodiments, an antisense agent is a nucleic acid therapeutic that acts by reducing the expression of a target gene, thereby reducing the expression of the polypeptide encoded by the target gene. Exemplary antisense agents that reduce or inhibit the expression of ALAS1 include an antisense strand that comprises, or consists of, the antisense sequence of AD-60489, AD-60519, AD-61193, or AD-60819 (or a corresponding unmodified antisense sequence) (see, e.g., U.S. Pat. No. 10,119,143 which is incorporated by reference in its entirety).

One exemplary RNAi agent that reduces the expression of ALAS1, givosiran, is provided, for example, in U.S. Pat. No. 10,119,143, titled “Compositions and methods for inhibiting expression of the ALAS1 gene,” filed Apr. 4, 2016, incorporated herein by reference in its entirety for all purposes.

| RNAi agent | Sense strand (5′ to 3′) | Antisense strand (5′ to 3′) |
|---|---|---|
| Givosiran | csasgaaaGfaGfuGfuCfuCfaucuuaL96 | usAfsAfGfaUfgAfgAfcAfcUfcUfuUfcUfgsgsu |
| | (SEQ ID NO: 16) | (SEQ ID NO: 17) |

A, C, G, and U are adenosine-3′-phosphate, cytidine-3′-phosphate, guanosine-3′-phosphate, and uridine-3′-phosphate, respectively; a, c, g, and u are 2′-O-methyladenosine-3′-phosphate, 2′-O-methylcytidine-3′-phosphate, 2′-O-methylguanosine-3′-phosphate, and 2′-O-methyluridine-3′-phosphate, respectively; Af, Cf, Gf, and Uf are 2′-fluoroadenosine-3′-phosphate, 2′-fluorocytidine-3′-phosphate, 2′-fluoroguanosine-3′-phosphate, and 2′-fluorouridine-3′-phosphate, respectively; dT is 2′-deoxythymidine-3′-phosphate; s is a phosphorothioate linkage, and L96 is N-[tris(GalNAc-alkyl)-amidodecanoyl)]-4-hydroxyprolinol.

As used herein, a “subject diagnosed with AHP” is a subject who has been determined by a health care professional to meet one or both of a clinically determinative level of ALA and/or PBG and a mutation associated with AHP.

As used herein, “treatment”, treating”, and the like is understood as administration of a therapeutic agent to reduce the rate of progression of a disease or condition or to reduce at least one sign or symptom in a subject suffering from a disease. In certain embodiments, a sign of a disease can be a change in a biomarker from a healthy reference level prior to the development of overt symptoms of the disease. Natural history studies and clinical trials of AHP have demonstrated disease progression in the absence of treatment.

As used herein, “detection of a protein”, “detection of a biomarker”, and the like are understood as detection of a protein, or a sufficiently large fragment of the protein, to determine the identity of the protein by the method used, e.g., immunological method, chromatography method. In certain embodiments, detection of a protein can include detection of a one or more isoforms of a protein present in a subject when no distinction is made among the various isoforms. In certain embodiments, the detection method is a clinically accepted or validated method.

### Biomarkers for the Diagnosis and Management of AHP

Plasma proteomics and the identification of minimally invasive biomarkers is emerging as an integral part of modern drug discovery and clinical development. In an effort to leverage this approach, the plasma proteomes of AHP patients were investigated in a clinical proteomic study of AHP. The proteomics approach demonstrated that 212 plasma proteins of 1196 proteins studied were significantly different between healthy controls and patients with AHP. Of those 212 plasma proteins, three proteins with the greatest effect size difference between AHP patients and healthy controls include APLP1, KIM1, and MMP7 (FIGS. 1A-1B). Two of the three proteins with the greatest effect size between AHP patients and healthy controls (KIM1, MMP7) are biomarkers of renal injury. Additional renal injury biomarkers were explored based on measured plasma proteins and previous characterization of plasma levels and renal injury. Three additional renal injury biomarkers (NGAL, CST3, CHI3L1) showed significant elevations in patients with AHP.

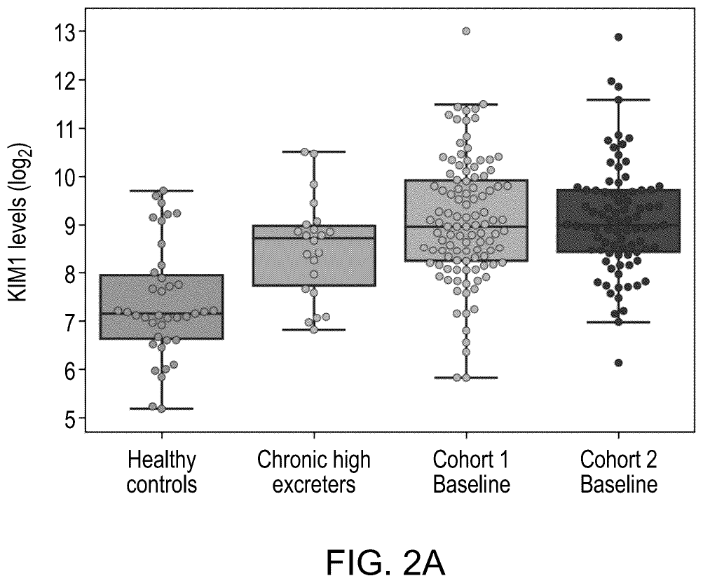

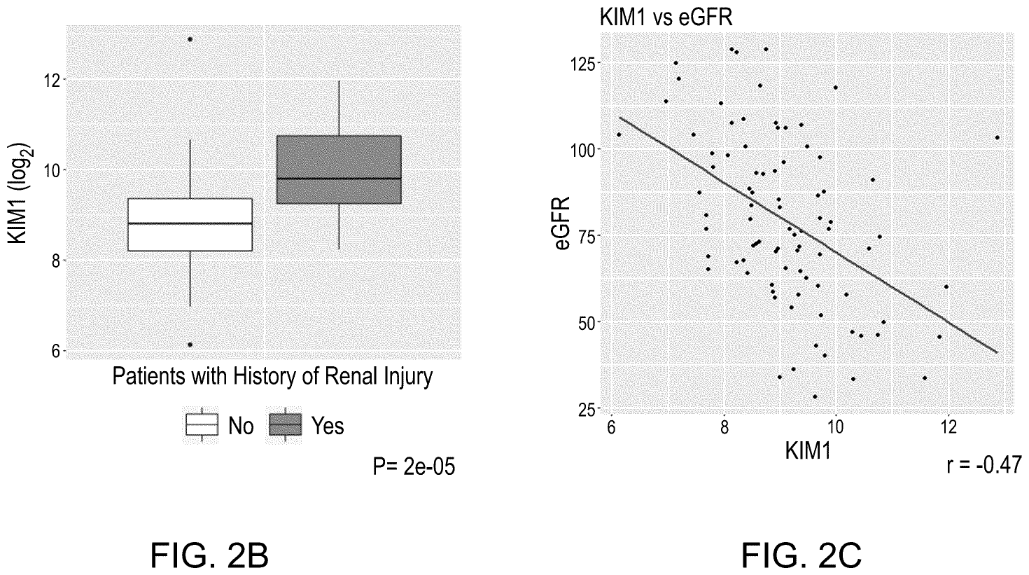

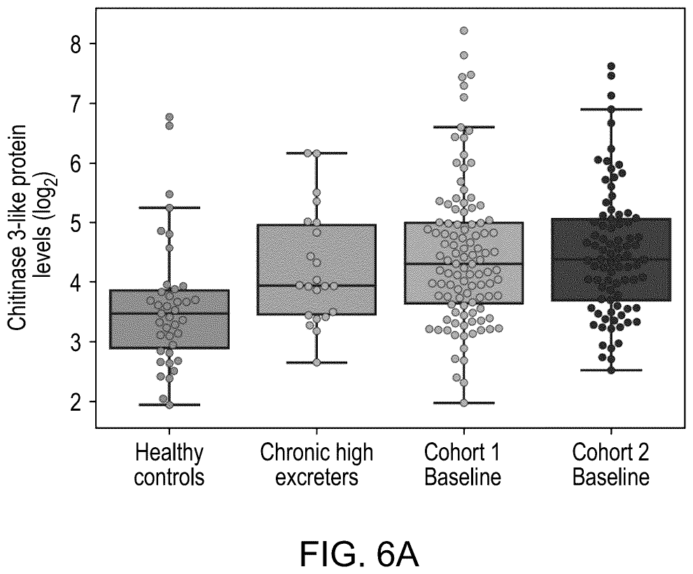

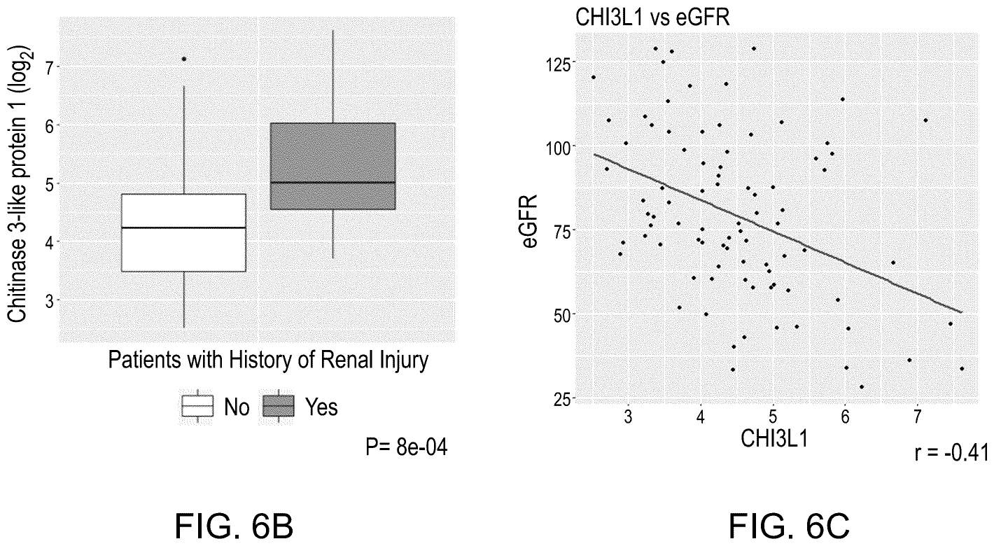

The demonstration that plasma levels of renal injury biomarkers are significantly elevated in AHP patients is relevant to diagnosis, treatment, and monitoring of AHP progression. The results are particularly compelling due to finding that these biomarkers are elevated in AHP patients with recurring acute attacks as well as chronic high excreters (CHE) (FIGS. 2A, 3A, 4A, 5A, and 6A).

A correlation was observed between renal injury biomarker levels and eGFR in patients with AHP (FIGS. 2B, 3B, 4B, 5B, and 6B). Glomerular filtration rate (GFR) is a measure of kidney function. Testing for actual GFR can be a complicated and lengthy procedure, which is why an estimate of GFR (eGFR) is usually calculated. Accurate estimates of GFR are important for early identification of kidney disease, which often has no symptoms until just before the kidneys fail. The correlation between eGFR and renal injury biomarker levels in patients with AHP is relevant to severity and disease progression of AHP. A correlation was also observed between renal injury biomarker levels and AHP patients with a history or prior diagnosis of renal failure and impairment.

As demonstrated herein, renal injury biomarker levels are elevated in AHP patients. In addition to monitoring disease regression upon treatment, renal injury protein levels may serve as a potential biomarker in other aspects of AHP disease. If levels of renal injury biomarkers increase during AHP early disease development, the renal injury biomarkers may serve as a prognostic indicator for the incidence of symptomatic disease, e.g., an acute attack as compared to CHE. Additionally, the correlation of renal injury biomarker levels with eGFP indicates that the renal injury biomarker levels may correlate with severity of the AHP disease. Lastly, renal injury biomarkers may potentially play an important role for distinguishing between effectiveness of various treatments. The present study was based on patients with AHP recurring acute attacks as well as CHE. Therefore, the presence of elevated renal injury biomarkers is not limited to patients exhibiting exclusively an acute attack porphyria.

In certain embodiments, the biomarker is chosen from one or more (e.g., 2, 3, 4, 5, or all) of KIM1, APLP1, MMP7, NGAL, CST3, or CHI3L1. In some embodiments, the biomarker comprises KIM1. In some embodiments, the biomarker comprises APLP1. In some embodiments, the biomarker comprises MMP7. In some embodiments, the biomarker comprises NGAL. In some embodiments, the biomarker comprises CST3. In some embodiments, the biomarker comprises CHI3L1. In some embodiments, the biomarker is a renal injury biomarker chosen from one or more (e.g., 2, 3, 4, or all) of KIM1, MMP7, NGAL, CST3, or CHI3L1.

In some embodiments, the levels of a plurality of biomarkers are determined. In some embodiments, the biomarkers comprise KIM1 and APLP1. In some embodiments, the biomarkers comprise KIM1 and MMP7. In some embodiments, the biomarkers comprise KIM1 and NGAL. In some embodiments, the biomarkers comprise KIM1 and CST3. In some embodiments, the biomarkers comprise KIM1 and CHI3L1. In some embodiments, the biomarkers comprise APLP1 and MMP7. In some embodiments, the biomarkers comprise APLP1 and NGAL. In some embodiments, the biomarkers comprise APLP1 and CST3. In some embodiments, the biomarkers comprise APLP1 and CHI3L1. In some embodiments, the biomarkers comprise MMP7 and NGAL. In some embodiments, the biomarkers comprise MMP7 and CST3. In some embodiments, the biomarkers comprise MMP7 and CHI3L1. In some embodiments, the biomarkers comprise NGAL and CST3. In some embodiments, the biomarkers comprise NGAL and CHI3L1. In some embodiments, the biomarkers comprise CST3 and CHI3L1.

In some embodiments, the biomarkers comprise KIM1, MMP7, and APLP1. In some embodiments, the biomarkers comprise KIM1, MMP7, and NGAL. In some embodiments, the biomarkers comprise KIM1, MMP7, and CST3. In some embodiments, the biomarkers comprise KIM1, MMP7, and CHI3L1. In some embodiments, the biomarkers comprise KIM1, NGAL, and CST3. In some embodiments, the biomarkers comprise KIM1, NGAL, and CHI3L1. In some embodiments, the biomarkers comprise KIM1, CST3, and CHI3L1. In some embodiments, the biomarkers comprise APLP1, MMP7, and NGAL. In some embodiments, the biomarkers comprise APLP1, MMP7, and CST3. In some embodiments, the biomarkers comprise APLP1, MMP7, and CHI3L1. In some embodiments, the biomarkers comprise APLP1, NGAL, and CST3. In some embodiments, the biomarkers comprise APLP1, NGAL, and CHI3L1. In some embodiments, the biomarkers comprise APLP1, CST3, and CHI3L1. In some embodiments, the biomarkers comprise MMP7, NGAL, and CST3. In some embodiments, the biomarkers comprise MMP7, NGAL, and CHI3L1. In some embodiments, the biomarkers comprise MMP7, CST3, and CHI3L1. In some embodiments, the biomarkers comprise NGAL, CST3, and CHI3L1.

In some embodiments, the biomarkers comprise KIM1, APLP1, MMP7, and NGAL. In some embodiments, the biomarkers comprise KIM1, APLP1, MMP7, and CST3. In some embodiments, the biomarkers comprise KIM1, APLP1, MMP7, and CHI3L1. In some embodiments, the biomarkers comprise KIM1, APLP1, NGAL, and CST3. In some embodiments, the biomarkers comprise KIM1, APLP1, NGAL, and CHI3L1. In some embodiments, the biomarkers comprise KIM1, APLP1, CST3, and CHI3L1. In some embodiments, the biomarkers comprise KIM1, MMP7, NGAL, and CST3. In some embodiments, the biomarkers comprise KIM1, MMP7, NGAL, and CHI3L1. In some embodiments, the biomarkers comprise KIM1, MMP7, CST3, and CHI3L1. In some embodiments, the biomarkers comprise KIM1, NGAL, CST3, and CHI3L1. In some embodiments, the biomarkers comprise MMP7, NGAL, CST3, and CHI3L1. In some embodiments, the biomarkers comprise APLP1, MMP7, NGAL, and CST3. In some embodiments, the biomarkers comprise APLP1, MMP7, NGAL, and CHI3L1. In some embodiments, the biomarkers comprise APLP1, MMP7, CST3, and CHI3L1. In some embodiments, the biomarkers comprise APLP1, NGAL, CST3, and CHI3L1.

In some embodiments, the biomarkers comprise KIM1, APLP1, MMP7, NGAL, and CST3. In some embodiments, the biomarkers comprise KIM1, APLP1, MMP7, NGAL, and CHI3L1. In some embodiments, the biomarkers comprise KIM1, APLP1, MMP7, CST3, and CHI3L1. In some embodiments, the biomarkers comprise KIM1, APLP1, NGAL, CST3, and CHI3L1. In some embodiments, the biomarkers comprise KIM1, MMP7, NGAL, CST3, and CHI3L1. In some embodiments, the biomarkers comprise APLP1, MMP7, NGAL, CST3, and CHI3L1.

In some embodiments, the biomarkers comprise KIM1, APLP1, MMP7, NGAL, CST3, and CHI3L1.

In some embodiments, the biomarkers further comprise KIM1.

In some embodiments, the biomarkers further comprise APLP1.

In some embodiments, the biomarkers further comprise MMP7.

In some embodiments, the biomarkers further comprise NGAL.

In some embodiments, the biomarkers further comprise CST3.

In some embodiments, the biomarkers further comprise CHI3L1.

### Biomarkers for Managing the Progression of AHP

Genetic testing is available for identification of subjects who have an AHP associated mutation. This disclosure provides biomarkers, e.g., renal injury biomarkers, to determine when a subject with a predisposition to AHP, e.g., a subject with a genetic predisposition or a subject with elevated ALA and/or PBG, may be treated with an agent that reduces the expression of ALAS1.

In certain embodiments, the biomarker is chosen from one or more (e.g., 2, 3, 4, 5, or all) of KIM1, APLP1, MMP7, NGAL, CST3, or CHI3L1. In some embodiments, the biomarker comprises KIM1. In some embodiments, the biomarker comprises APLP1. In some embodiments, the biomarker comprises MMP7. In some embodiments, the biomarker comprises NGAL. In some embodiments, the biomarker comprises CST3. In some embodiments, the biomarker comprises CHI3L1. In some embodiments, the biomarker is a renal injury biomarker chosen from one or more (e.g., 2, 3, 4, or all) of KIM1, MMP7, NGAL, CST3, or CHI3L1.

In some embodiments, the levels of a plurality of biomarkers are determined. In some embodiments, the biomarkers comprise KIM1 and APLP1. In some embodiments, the biomarkers comprise KIM1 and MMP7. In some embodiments, the biomarkers comprise KIM1 and NGAL. In some embodiments, the biomarkers comprise KIM1 and CST3. In some embodiments, the biomarkers comprise KIM1 and CHI3L1. In some embodiments, the biomarkers comprise APLP1 and MMP7. In some embodiments, the biomarkers comprise APLP1 and NGAL. In some embodiments, the biomarkers comprise APLP1 and CST3. In some embodiments, the biomarkers comprise APLP1 and CHI3L1. In some embodiments, the biomarkers comprise MMP7 and NGAL. In some embodiments, the biomarkers comprise MMP7 and CST3. In some embodiments, the biomarkers comprise MMP7 and CHI3L1. In some embodiments, the biomarkers comprise NGAL and CST3. In some embodiments, the biomarkers comprise NGAL and CHI3L1. In some embodiments, the biomarkers comprise CST3 and CHI3L1.

In some embodiments, the biomarkers comprise KIM1, MMP7, and APLP1. In some embodiments, the biomarkers comprise KIM1, MMP7, and NGAL. In some embodiments, the biomarkers comprise KIM1, MMP7, and CST3. In some embodiments, the biomarkers comprise KIM1, MMP7, and CHI3L1. In some embodiments, the biomarkers comprise KIM1, NGAL, and CST3. In some embodiments, the biomarkers comprise KIM1, NGAL, and CHI3L1. In some embodiments, the biomarkers comprise KIM1, CST3, and CHI3L1. In some embodiments, the biomarkers comprise APLP1, MMP7, and NGAL. In some embodiments, the biomarkers comprise APLP1, MMP7, and CST3. In some embodiments, the biomarkers comprise APLP1, MMP7, and CHI3L1. In some embodiments, the biomarkers comprise APLP1, NGAL, and CST3. In some embodiments, the biomarkers comprise APLP1, NGAL, and CHI3L1. In some embodiments, the biomarkers comprise APLP1, CST3, and CHI3L1. In some embodiments, the biomarkers comprise MMP7, NGAL, and CST3. In some embodiments, the biomarkers comprise MMP7, NGAL, and CHI3L1. In some embodiments, the biomarkers comprise MMP7, CST3, and CHI3L1. In some embodiments, the biomarkers comprise NGAL, CST3, and CHI3L1.

In some embodiments, the biomarkers comprise KIM1, APLP1, MMP7, and NGAL. In some embodiments, the biomarkers comprise KIM1, APLP1, MMP7, and CST3. In some embodiments, the biomarkers comprise KIM1, APLP1, MMP7, and CHI3L1. In some embodiments, the biomarkers comprise KIM1, APLP1, NGAL, and CST3. In some embodiments, the biomarkers comprise KIM1, APLP1, NGAL, and CHI3L1. In some embodiments, the biomarkers comprise KIM1, APLP1, CST3, and CHI3L1. In some embodiments, the biomarkers comprise KIM1, MMP7, NGAL, and CST3. In some embodiments, the biomarkers comprise KIM1, MMP7, NGAL, and CHI3L1. In some embodiments, the biomarkers comprise KIM1, MMP7, CST3, and CHI3L1. In some embodiments, the biomarkers comprise KIM1, NGAL, CST3, and CHI3L1. In some embodiments, the biomarkers comprise MMP7, NGAL, CST3, and CHI3L1. In some embodiments, the biomarkers comprise APLP1, MMP7, NGAL, and CST3. In some embodiments, the biomarkers comprise APLP1, MMP7, NGAL, and CHI3L1. In some embodiments, the biomarkers comprise APLP1, MMP7, CST3, and CHI3L1. In some embodiments, the biomarkers comprise APLP1, NGAL, CST3, and CHI3L1.

In some embodiments, the biomarkers comprise KIM1, APLP1, MMP7, NGAL, and CST3. In some embodiments, the biomarkers comprise KIM1, APLP1, MMP7, NGAL, and CHI3L1. In some embodiments, the biomarkers comprise KIM1, APLP1, MMP7, CST3, and CHI3L1. In some embodiments, the biomarkers comprise KIM1, APLP1, NGAL, CST3, and CHI3L1. In some embodiments, the biomarkers comprise KIM1, MMP7, NGAL, CST3, and CHI3L1. In some embodiments, the biomarkers comprise APLP1, MMP7, NGAL, CST3, and CHI3L1.

In some embodiments, the biomarkers comprise KIM1, APLP1, MMP7, NGAL, CST3, and CHI3L1.

In some embodiments, the biomarkers further comprise KIM1.

In some embodiments, the biomarkers further comprise APLP1.

In some embodiments, the biomarkers further comprise MMP7.

In some embodiments, the biomarkers further comprise NGAL.

In some embodiments, the biomarkers further comprise CST3.

In some embodiments, the biomarkers further comprise CHI3L1.

In certain embodiments, after the subject is identified as having an AHP associated mutation, the subject is routinely monitored for an increase in biomarker (e.g., renal biomarker) levels as compared to a reference level, either a population control or a biomarker (e.g., renal biomarker) level for the same subject. An increase in the biomarker (e.g., renal biomarker) in the subject is an indicator treatment of the subject, e.g., with an agent that reduces the expression of ALAS1 should be initiated. In certain embodiments, the subject is also routinely monitored for the development of a sign or symptom of AHP. In certain embodiments, the subject is also monitored for the level of one or more of ALA and PBG, wherein an increase in the level of a biomarker (e.g., renal biomarker) as compared to a reference level when the beta coefficient is positive is indicative of worsening AHP, and a decrease in the level of a biomarker (e.g., renal biomarker) as compared to a reference level when the beta coefficient is negative is indicative of worsening AHP.

Monitoring of biomarker (e.g., renal biomarker) levels can also be used to determine if AHP is progressing in the subject, where an increase in the level of the biomarker (e.g., renal biomarker) in the subject is indicative of progressive AHP. Progression can be monitored by further determining the level of one or more of ALA and PBG, wherein an increase in the level of a biomarker (e.g., renal biomarker) as compared to a reference level when the beta coefficient is positive is indicative of worsening AHP, and a decrease in the level of a biomarker (e.g., renal biomarker) as compared to a reference level when the beta coefficient is negative is indicative of worsening AHP.

### Establishing Reference Standards of Biomarker Levels

The disclosure provides the steps of measuring one or more biomarker (e.g., protein biomarker) levels in a subject and comparison of a biomarker level to its corresponding reference level to determine if there is a difference between the biomarker level and its corresponding reference level. It is understood that the method for determining the level of the biomarker in the sample and the method by which the reference level was determined for the reference level should be the same. Further, it is understood that the change from the reference level may be a change in a defined concentration of a biomarker in a sample, e.g., pg/ml of sample. Alternatively, the change can be a relative amount, a change in percent of the reference sample, e.g., at least 150% or at least 200% of the reference sample. The change in the level should be statistically significant. Methods to determine biomarker levels are typically performed in vitro, often in a clinical lab setting when used for diagnostic methods and methods used to select a treatment for a subject.

Commercially available kits are available to determine the level of some biomarkers, e.g., NT-proBNP and troponin I. Levels of these markers can be performed in clinical laboratories using commercially available diagnostic tests, e.g., using chemiluminescence assays (Roche Diagnostic Cobas, Indianapolis, IN, USA for NT-proBNP; Siemens Centaur XP, Camberley, Surrey, UK for troponin I). In such cases, the reference level of the biomarker, and in embodiments an appropriate control, can be provided by the kit manufacturer.

Previous studies demonstrate that generally no single reference level is appropriate for all subjects. Instead, an appropriate age and gender matched control reference standard can be selected for comparison to population-based controls. Such considerations are understood in the art.

When an earlier time point for a subject is used as a reference level, a sufficient interval to allow for a change in biomarker level needs to be provided. Changes in renal injury biomarker levels were observed between day 0 and one or more subsequent time point, both in control subjects with increasing levels of the renal injury biomarker, and in treated subjects with decreasing levels. A sufficient interval for a change in renal injury biomarker level may be determined for each renal injury biomarker. It is expected that short intervals, e.g., 6 months, 3 months, 2 months, 1 month or even less than one month, can be sufficient to observe a change in renal injury biomarker level in a subject, depending on the particular biomarker, disease state, and rate of progression in the subject.

### Signs and Symptoms of Porphyria

The methods disclosed herein can further include monitoring subjects for one or more signs or symptoms indicative of AHP or progression of AHP including, but not limited to, abdominal pain, limb, back, or chest pain, nausea, vomiting, confusion, anxiety, seizures, constipation, diarrhea, dark reddish urine, or any combination thereof. Comorbidities indicative of AHP or AHP progression that may be monitored include, for example, hypertension, chronic kidney disease, and combinations thereof. The development or progression of one or more of the signs or symptoms, in the context of an elevated renal injury biomarker level as compared to a reference level, is further diagnostic of AHP.

Nucleic Acid Therapeutics that Reduce the Expression of ALAS1

In some embodiments, the methods described herein involve use of a nucleic acid therapeutic agent (e.g., an RNAi agent) that reduces expression of ALAS1. In certain embodiments, expression of an ALAS1 gene is reduced or inhibited using an ALAS1-specific iRNA.

Described herein are compositions and methods that effect the RNA induced silencing complex (RISC)-mediated cleavage of RNA transcripts of the ALAS1 gene, such as in a cell or in a subject (e.g., in a mammal, such as a human subject).

The iRNAs included in the compositions featured herein encompass a dsRNA having an RNA strand (the antisense strand) having a region, e.g., a region that is 30 nucleotides or less, generally 19-24 nucleotides in length, that is substantially complementary to at least part of an mRNA transcript of an ALAS1 gene (e.g., a mouse or human ALAS1 gene) (also referred to herein as an “ALAS1-specific iRNA”). Alternatively, or in combination, iRNAs encompass a dsRNA having an RNA strand (the antisense strand) having a region that is 30 nucleotides or less, generally 19-24 nucleotides in length, that is substantially complementary to at least part of an mRNA transcript of an ALAS1 gene (e.g., a human variant 1 or 2 of an ALAS1 gene) (also referred to herein as a “ALAS1-specific iRNA”).

In embodiments, the iRNA (e.g., dsRNA) described herein comprises an antisense strand having a region that is substantially complementary to a region of a human ALAS1. In embodiments, the human ALAS1 has the sequence of NM_000688.4 (SEQ ID NO: 11). In embodiments, the human ALAS1 has the sequence of NM_199166.1 (SEQ ID NO: 12). In embodiments, the antisense sequence of the iRNA (e.g., dsRNA) targets within the region 871 to 895 (plus or minus 5, 4, 3, 2, or 1 nucleotides in either or both directions on the 5′ and/or 3′ end) on the ALAS1 transcript NM_000688.4. In embodiments, the antisense sequence targets the nucleotides 871 to 893, 871 to 892, or 873 to 895 on the ALAS1 transcript NM_000688.4. In embodiments, the antisense sequence comprises or consists of a sequence that is fully complementary or substantially complementary to nucleotides 871 to 893, 871 to 892, or 873 to 895 on the ALAS1 transcript NM_000688.4.

In one aspect, a double-stranded ribonucleic acid (dsRNA) for inhibiting expression of ALAS1 is provided, wherein said dsRNA comprises a sense strand and an antisense strand, the antisense strand comprising a region of complementarity to an ALAS1 RNA transcript, which antisense strand comprises at least 15 (e.g., at least 16, 17, 18, 19, 20, 21, 22, or 23) contiguous nucleotides differing by no more than 3, 2 or 1 nucleotides from the sequence of UAAGAUGAGACACUCUUUCUGGU (SEQ ID NO: 13) or UAAGAUGAGACACUCTUUCUGGU (SEQ ID NO: 14). In embodiments, the antisense strand comprises the sequence of UAAGAUGAGACACUCUUUCUGGU (SEQ ID NO: 13) or UAAGAUGAGACACUCTUUCUGGU (SEQ ID NO: 14). In embodiments, the sense strand comprises the sequence of CAGAAAGAGUGUCUCAUCUUA (SEQ ID NO: 15). In embodiments, one or more nucleotides of the antisense strand and/or sense strand are modified as described herein.

In some embodiments, the dsRNA is effective to suppress the liver level of ALAS1 mRNA, e.g., to achieve silencing of at least 10%, 20%, 30%, 40%, 50%, 60%, 70%, or 80% (e.g., such that ALAS1 mRNA levels are decreased to 90% or less, 80% or less, 70% or less, 60% or less, 50% or less, 40% or less, 30% or less, or 20% or less of a control level of liver ALAS1 mRNA, e.g., the level in an untreated individual or group of individuals, e.g., an individual or group of individuals treated with PBS only).

In some embodiments, the dsRNA is effective to suppress the circulating level of ALAS1 mRNA, e.g., to achieve silencing of at least 10%, 20%, 30%, 40%, 50%, 60%, 70%, or 80% (e.g., such that ALAS1 mRNA levels are decreased to 90% or less, 80% or less, 70% or less, 60% or less, 50% or less, 40% or less, 30% or less, or 20% or less of a control level of circulating ALAS1 mRNA, e.g., the level prior to treatment with the dsRNA, or the level in an untreated individual or group of individuals).

The iRNA molecules featured herein can include naturally occurring nucleotides or can include at least one modified nucleotide. In embodiments, the at least one modified nucleotide include one or more of a modification on the nucleotide chosen from the group consisting of a locked nucleic acid (LNA), an acyclic nucleotide, a hexitol or hexose nucleic acid (HNA), a cyclohexene nucleic acid (CeNA), 2′-methoxyethyl, 2′-O-alkyl, 2′-O-allyl, 2′-C-allyl, 2′-fluoro, 2′-deoxy, 2′-hydroxyl, or any combination thereof. In one embodiment, the at least one modified nucleotide includes, but is not limited to a 2′-O-methyl modified nucleotide, 2′-fluoro modified nucleotide, a nucleotide having a 5′-phosphorothioate group, and a terminal nucleotide linked to a ligand, e.g., an N-acetylgalactosamine (GalNAc) or a cholesteryl derivative.

Alternatively, the modified nucleotide may be chosen from the group of: a 2′-deoxy-2′-fluoro modified nucleotide, a 2′-deoxy-modified nucleotide, a locked nucleotide, an acyclic nucleotide, an abasic nucleotide, 2′-amino-modified nucleotide, 2′-alkyl-modified nucleotide, morpholino nucleotide, a phosphoramidate, and a non-natural base comprising nucleotide.

In some embodiments, the region of complementarity is at 5 least 17 nucleotides in length. In some embodiments, the region of complementarity is between 19 and 21 nucleotides in length. In some embodiments, the region of complementarity is 19 nucleotides in length. In some embodiments, each strand is no more than 30 nucleotides in length.

In some embodiments, at least one strand comprises a 3′ overhang of at least 1 nucleotide. In embodiments, the antisense strand comprises a 3′ overhang of at least 1 nucleotide.

In some embodiments, at least one strand comprises a 3′ overhang of at least 2 nucleotides. In embodiments, the antisense strand comprises a 3′ overhang of at least 2 nucleotides. In embodiments, the antisense strand comprises a 3′ overhang of 2 nucleotides.

In one embodiment, an iRNA as described herein targets a wildtype ALAS1 RNA transcript variant, and in another embodiment, the iRNA targets a mutant transcript (e.g., an ALAS1 RNA carrying an allelic variant). For example, an iRNA featured in the disclosure can target a polymorphic variant, such as a single nucleotide polymorphism (SNP), of ALAS1. In another embodiment, the iRNA targets both a wildtype and a mutant ALAS1 transcript. In yet another embodiment, the iRNA targets a particular transcript variant of ALAS1 (e.g., human ALAS1 variant 1). In yet another embodiment, the iRNA agent targets multiple transcript variants (e.g., both variant 1 and variant 2 of human ALAS1).

In one embodiment, an iRNA featured in the disclosure targets a non-coding region of an ALAS1 RNA transcript, such as the 5′ or 3′ untranslated region of a transcript. In some embodiments, an iRNA as described herein is in the form of a conjugate, e.g., a carbohydrate conjugate, which may serve as a targeting moiety and/or ligand, as described herein. In one embodiment, the conjugate is attached to the 3′ end of the sense strand of the dsRNA. In some embodiments, the conjugate is attached via a linker, e.g., via a bivalent or trivalent branched linker.

In some embodiments, the conjugate comprises one or more N-acetylgalactosamine (GalNAc) derivatives. Such a conjugate is also referred to herein as a GalNAc conjugate. In some embodiments, the conjugate targets the RNAi agent to a particular cell, e.g., a liver cell, e.g., a hepatocyte. The GalNAc derivatives can be attached via a linker, e.g., a bivalent or trivalent branched linker. In some embodiments, the RNAi agent is attached to the carbohydrate conjugate via a linker.

In some embodiments, the dsRNA comprises a sense strand and an antisense strand, the antisense strand comprising a region of complementarity to an ALAS1 RNA transcript, wherein the sense strand comprises the sequence and all of the modifications of csasgaaaGfaGfuGfuCfuCfaucuuaL96 (SEQ ID NO: 16), and wherein the antisense strand comprises the sequence and all of the modifications of usAfsAfGfaUfgAfgAfcAfcUfcUfuUfcUfgsgsu (SEQ ID NO: 17),
wherein c, a, g, u=2′-OMe ribonucleosides; Af, Cf, Gf, Uf=2′F ribonucleosides; s=phosphorothioate, and
wherein

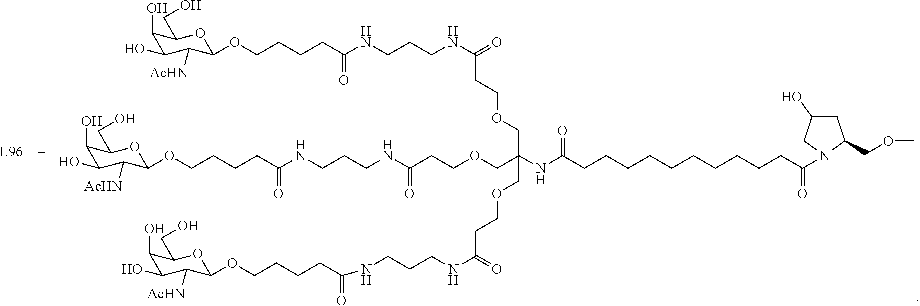 [SMILES: COC[C@@H]1CC(O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the dsRNA comprises a duplex region which is 21-23 nucleotide pairs in length. In some embodiments, at least one strand comprises a 3′ overhang of at least 2 nucleotides. In some embodiments, each strand is no more than 26 nucleotides in length.

In some embodiments, the antisense strand consists of the sequence of usAfsAfGfaUfgAfgAfcAfcUfcUfuUfcUfgsgsu (SEQ ID NO: 17). In some embodiments, the sense strand consists of the sequence of csasgaaaGfaGfuGfuCfuCfaucuuaL96 (SEQ ID NO: 16).

In some embodiments, the sense strand consists of the sequence of csasgaaaGfaGfuGfuCfuCfaucuuaL96 (SEQ ID NO: 16), and the antisense strand consists of the sequence of usAfsAfGfaUfgAfgAfcAfcUfcUfuUfcUfgsgsu (SEQ ID NO: 17).

In some embodiments, the dsRNA comprises a sense strand and an antisense strand, the antisense strand comprising a region of complementarity to an ALAS1 RNA transcript, wherein said dsRNA is in the form of a conjugate having the structure of:

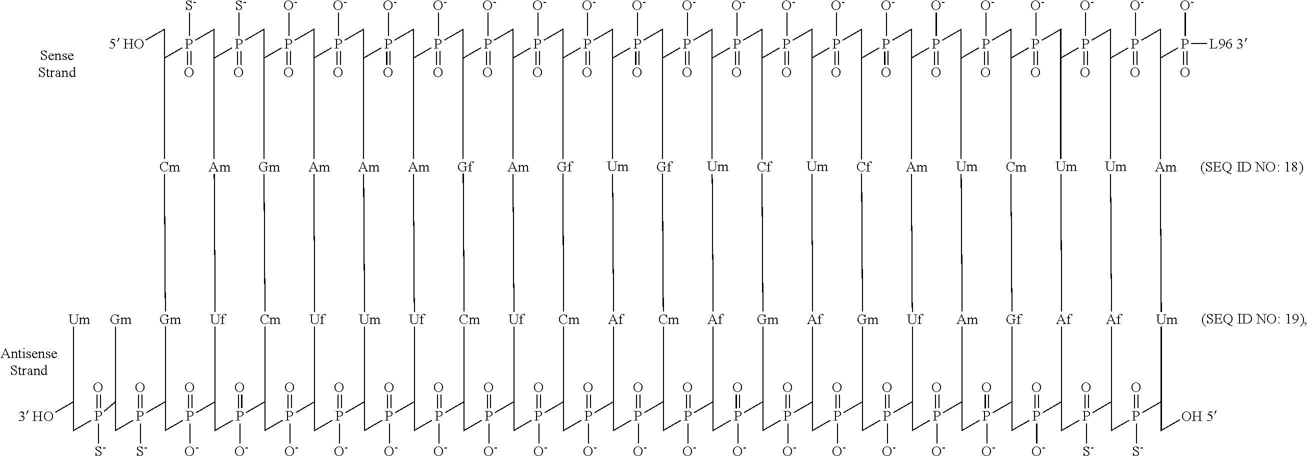

or a pharmaceutically acceptable salt thereof,
wherein Af, Cf, Gf, Uf=2′F ribonucleosides; Am, Cm, Gm, Um=2′-OMe ribonucleosides;

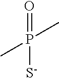

=phosphorothioate;

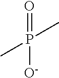

=phosphodiester, and
wherein

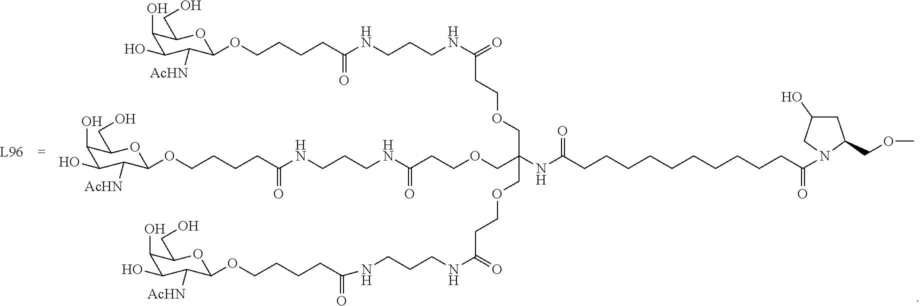 [SMILES: COC[C@@H]1CC(O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the dsRNA comprises a duplex region which is 21 nucleotide pairs in length. In some embodiments, the antisense strand comprises a 3′ overhang of two nucleotides.

Other exemplary nucleic acid therapeutics that reduce that reduce the expression of ALAS1 are disclosed in International Application Publication Nos. WO2013/155204 and WO2015/051318, U.S. Pat. Nos. 9,133,461, 9,631,193, 10,400,239, 10,119,143, 10,125,364 and 11,028,392, and U.S. Application Publication Nos. US2013/0281511, US2015/0111841, US2016/0115476, US2016/0244766, US2018/0037886, US2019/0144870, US2019/0218549, US2020/0181614, and US2021/0087558, the contents of each of the aforesaid publications are incorporated by reference in their entirety.

In an aspect provided herein is a composition, e.g., a pharmaceutical composition, that includes one or more of the iRNAs described herein and a pharmaceutically acceptable carrier or delivery vehicle. In one embodiment, the composition is used for inhibiting the expression of an ALAS1 gene in an organism, generally a human subject. In one embodiment, the composition is used for treating a porphyria, e.g., AHP, e.g., AIP.

In embodiments of the pharmaceutical compositions described herein, the iRNA (e.g., dsRNA) is administered in an unbuffered solution. In embodiments, the unbuffered solution is saline or water, e.g., water for injection.

In some embodiments, the pharmaceutical composition comprises the iRNA and water for injection. In embodiments, the composition comprises about 100 to 300 mg/mL, e.g., 200 mg/mL, of the iRNA. In embodiments, the composition has a pH of 6.0-7.5, e.g., about 7.0. In embodiments, the composition is for subcutaneous injection. In embodiments, the pharmaceutical composition is packaged in a container (e.g., a glass vial, e.g., a 2 mL glass vial) at a volume of about 0.3 to 1 mL, e.g., 0.55 mL.

In embodiments of the pharmaceutical compositions described herein, the iRNA (e.g., dsRNA is administered with a buffer solution. In embodiments, the buffer solution comprises acetate, citrate, prolamine, carbonate, or phosphate or any combination thereof. In embodiments, the buffer solution is phosphate buffered saline (PBS).

In embodiments of the pharmaceutical compositions described herein, the iRNA (e.g., dsRNA) is targeted to hepatocytes.

In embodiments of the pharmaceutical compositions described herein, the composition is administered intravenously. In embodiments of the pharmaceutical compositions described herein, the composition is administered subcutaneously.

In some embodiments, the dsRNA is administered monthly at a dose of 0.01 mg/kg to 5 mg/kg or 1 mg/kg to 2.5 mg/kg bodyweight of the subject. In some embodiments, the dsRNA is administered monthly at a dose of 2.5 mg/kg bodyweight of the subject.

In embodiments, a pharmaceutical composition comprises an iRNA (e.g., a dsRNA) described herein that comprises a ligand (e.g., a GalNAc ligand) that targets the iRNA (e.g., dsRNA) to hepatocytes.

In embodiments, a pharmaceutical composition comprises an iRNA (e.g., a dsRNA) described herein that comprises a ligand (e.g., a GalNAc ligand), and the pharmaceutical composition is administered subcutaneously. In embodiments, the ligand targets the iRNA (e.g., dsRNA) to hepatocytes.

In certain embodiments, a pharmaceutical composition, e.g., a composition described herein, includes a lipid formulation. In some embodiments, the RNAi agent is in an LNP formulation, e.g., a MC3 formulation. In some embodiments, the LNP formulation targets the RNAi agent to a particular cell, e.g., a liver cell, e.g., a hepatocyte. In embodiments, the lipid formulation is a LNP11 formulation. In embodiments, the composition is administered intravenously.

In another embodiment, the pharmaceutical composition is formulated for administration according to a dosage regimen described herein, e.g., not more than once every four weeks, not more than once every three weeks, not more than once every two weeks, or not more than once every week. In another embodiment, the administration of the pharmaceutical composition can be maintained for a month or longer, e.g., one, two, three, or six months, or one year or longer.

In another embodiment, a composition containing an iRNA featured in the disclosure, e.g., a dsRNA targeting ALAS1, is administered with a non-iRNA therapeutic agent, such as an agent known to treat a porphyria (e.g., AIP), or a symptom of a porphyria (e.g., pain). In another embodiment, a composition containing an iRNA featured in the disclosure, e.g., a dsRNA targeting AIP, is administered along with a non-iRNA therapeutic regimen, such as hemin or glucose (e.g., glucose infusion (e.g., IV glucose)). For example, an iRNA featured in the disclosure can be administered before, after, or concurrent with glucose, dextrose, or a similar treatment that serves to restore energy balance (e.g., total parenteral nutrition). An iRNA featured in the disclosure can also be administered before, after, or concurrent with the administration of a heme product (e.g., hemin, heme arginate, or heme albumin), and optionally also in combination with a glucose (e.g. IV glucose) or the like.

Typically, glucose administered for the treatment of a porphyria is administered intravenously (IV). Administration of glucose intravenously is referred to herein as “IV glucose.” However, alternative embodiments in which glucose is administered by other means are also encompassed.

In one embodiment, an ALAS1 iRNA is administered to a patient, and then the non-iRNA agent or therapeutic regimen (e.g., glucose and/or a heme product) is administered to the patient (or vice versa). In another embodiment, an ALAS1 iRNA and the non-iRNA therapeutic agent or therapeutic regimen are administered at the same time.

### Further Embodiments

Disclosed herein are methods of treating a human subject having, or at risk of having, AHP. The subject may have recurring acute attacks of AHP. The subject may be a chronic high excreter.

In some embodiments, the subject has not been diagnosed as having a porphyria. In some embodiments, the subject has not been diagnosed with AHP. For instance, the subject may not have been determined by a health care professional to meet one or both of a clinically determinative level of ALA and/or PBG and a mutation associated with AHP.

In some embodiments, the subject does not meet the diagnostic criteria for AHP. For instance, the subject may not meet one or both of (i) having a clinically determinative level of ALA and/or PBG and (ii) having a mutation associated with AHP.

In some embodiments, the subject has an elevated level of ALA and/or PBG. Elevated levels of ALA may be, for example, more than 35 μmol/L in a biological sample or more than 60 μmol/d in a 24-hour period. Elevated levels of PBG may be, for example, more than 8.8 μmol/d in a 24-hour period.

In some embodiments, the subject has a mutation associated with AHP. For example, the subject may have a specific AHP causative gene mutation (e.g., an HMBS mutation).

In some embodiments, the subject does not have a mutation associated with AHP. For example, the subject may not have a specific AHP causative gene mutation (e.g., an HMBS mutation).

In some embodiments, the subject has a history of renal impairment. For instance, the subject may have previously been diagnosed with a renal impairment. The subject may have recovered from the renal impairment. The subject may be suffering from the renal impairment.

In some embodiments, the subject has been diagnosed with a renal injury. For instance, the subject may have been diagnosed with chronic kidney disease or an acute kidney injury.

In some embodiments, the subject has a reduced estimated glomerular filtration rate (eGFR). The subject may have a stage 1, stage 2, stage 3, stage 4, or stage 5 eGFR. Stage 1 (eGFR of 90 or higher) indicates mild kidney damage, but kidneys are working well. Stage 2 (eGFR between 60 and 89) indicates an increase in kidney damage from stage 1, but the kidneys continue to function well. Stage 3 (eGFR between 30 and 59) indicates a decreased kidney function and the subject may experience symptoms. Stage 4 (eGFR between 15 and 29) is poor kidney function, with moderate to severe kidney damage. Stage 5 (eGFR below 15) is a sign of kidney failure associated with less than 15% kidney function.

In some embodiments, the level of the biomarker (e.g., renal injury biomarker) in the subject is increased by at least 2-fold, for example, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, least 15-fold, at least 16-fold, at least 17-fold, at least 18-fold, at least 19-fold, or at least 20-fold, compared to the reference level of the renal injury biomarker. The reference level may be a healthy control level or an earlier level in the same subject.

The level of the biomarker, e.g., renal injury biomarker may be determined in a subject sample selected from blood, plasma, serum, urine, or stool.

In some embodiments, the subject is being treated with a second therapeutic agent. The second therapeutic agent may be a therapeutic agent for AHP. For instance, the second therapeutic agent comprises a heme product (e.g., hemin, heme arginate, or heme albumin), glucose (e.g. IV glucose), dextrose, or combinations thereof. The second therapeutic agent may be a therapeutic agent for renal injury. For instance, the second therapeutic agent may be a therapeutic agent for chronic renal injury or acute renal injury. The second therapeutic agent may be a therapeutic agent for a symptom of AHP. The second therapeutic agent may be a therapeutic agent for a comorbidity of AHP.

The method may further comprise discontinuation of treatment with the second therapeutic agent when the subject has the elevated level of the biomarker, e.g., renal injury biomarker.

In some embodiments, the subject is further suffering from one or more symptoms associated with AHP. For instance, the subject may be suffering from abdominal pain, limb, back, or chest pain, nausea, vomiting, confusion, anxiety, seizures, constipation, diarrhea, dark reddish urine, or any combination thereof.

In some embodiments, the subject is further suffering from one or more comorbidities associated with AHP. For instance, the subject may be suffering from hypertension, chronic kidney disease, or both.

### EXAMPLES

### Example 1: Proteins Identified that Differ in AHP Patients Relative to Healthy Controls

An observational, case-controlled study was performed to identify changes in the proteome associated with AHP. The study compared proteomes of patients with AHP, chronic high excreters (CHE), and healthy controls. The results are shown in the graphs of FIGS. 1A-1B.

Proteomic analysis (OLINK® platform, Olink Proteomics Inc., Watertown, MA) was used to measure 1196 proteins in plasma from consenting AHP patients with recurrent acute attacks (>90% AIP) in a first cohort (n=108) (Natural History Study of AHP, Identifier No. NCT02240784), a second cohort (n=85) (Study to Evaluate the Efficacy and Safety of Givosiran in AHP patients, Phase 3, Identifier No. NCT03338816), and CHE patients (n=22) (Study of Givosiran in AIP, Phase 1, Identifier No. NCT02452372) at baseline. A separate cohort of healthy controls (n=39), age- and gender-matched to cohort 1 patients was also analyzed. Linear regression accounting for age, and sex was used to determine the proteins that were significantly different between AHP patients or CHE patients and healthy controls.

The proteomics assay plasma levels were analyzed to find a set of biomarkers indicative of AHP disease compared to healthy controls, symptomatic disease compared to CHE, and severity of AHP disease.

The patients from each of cohort 1 and cohort 2 were compared to healthy controls. A total of 212 plasma proteins were identified that differ in AHP patients relative to healthy controls. The 212 plasma proteins are plotted in the graphs of FIG. 1A (cohort 1) and FIG. 1B (cohort 2).

As shown in the data presented in FIGS. 1A-1B, from the 212 plasma proteins, matrix metalloproteinase-7 (MMP7), amyloid-like protein 1 (APLP1), and kidney injury molecule-1 (KIM1) were the proteins with the greatest effect size between AHP patients and healthy controls. The results were consistent between the two cohorts. KIM1 and MMP7, two biomarkers of renal injury, were identified as having large effect sizes (KIM1; 3.4-fold; p-value=8.0e-13) (MMP7; 5-fold; p-value=1.5e-25).

Accordingly, certain biomarkers, including, for example, KIM1, APLP1, and MMP7, may aid in diagnosing and managing kidney disease in patients with AHP.

### Example 2: Biomarkers of Renal Injury are Elevated in AHP Patients and CHE Relative to Healthy Controls

Biomarkers of renal injury were found to be significantly different between AHP patients and healthy controls in Example 1. Other biomarkers of renal injury were further studied. The results for five of these proteins are shown in the graphs of FIGS. 2A-2C, 3A-3C, 4A-4C, 5A-5C, and 6A-6C, respectively.

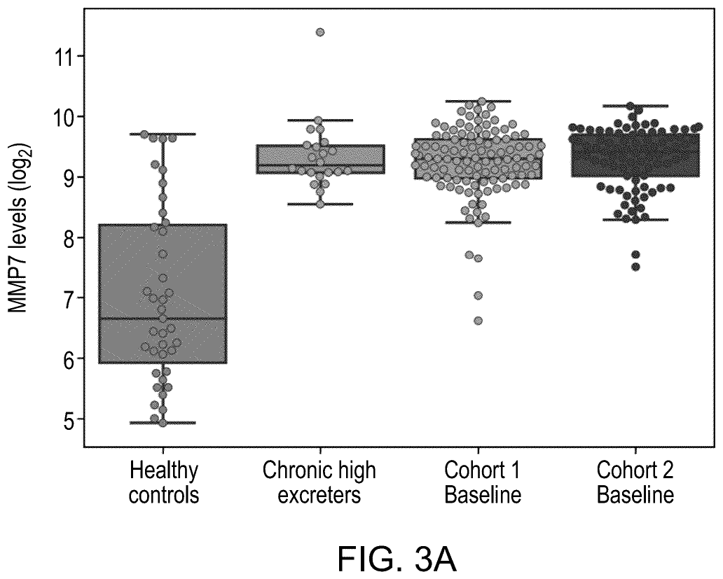

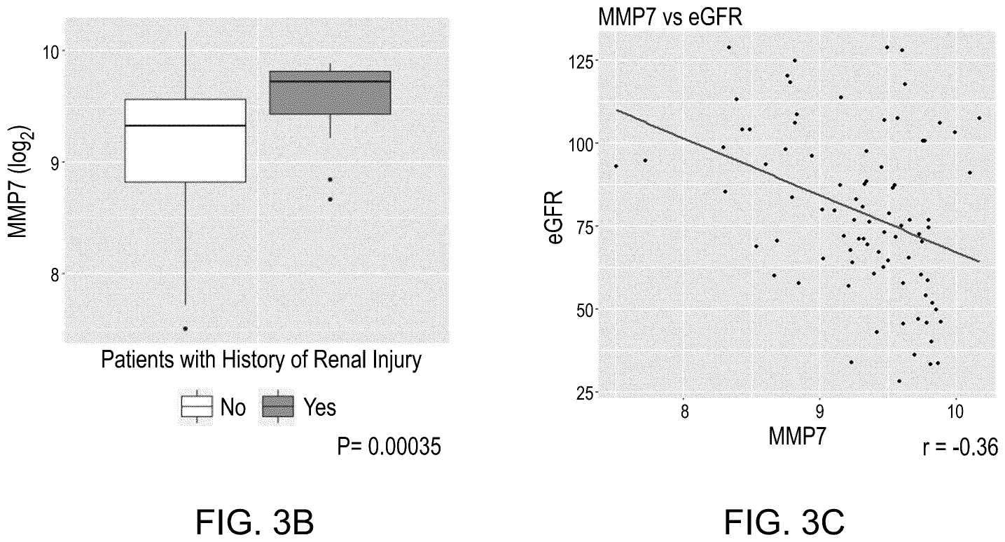

Plasma protein levels of the renal injury biomarkers were measured in the AHP patients from cohort 1 and cohort 2, as well as CHE patients, and compared to healthy controls. KIM1 and MMP7 are both significantly elevated in AHP patients from both cohorts and CHE relative to healthy control samples (p-values<0.01; FIGS. 2A and 3A, respectively).

KIM1 levels are further significantly elevated in cohort 2 patients with a history of renal failure and impairment (p-value 1e-5; 2.4×; FIG. 2B). KIM1 levels correlate well with eGFR (r=−0.47) and serum creatinine (r=0.47) (FIG. 2C). MMP7 is significantly elevated in patients with a history of renal failure and impairment (p-value 0.00035; FIG. 3B) and correlates somewhat with eGFR (r=−0.36) (FIG. 3C).

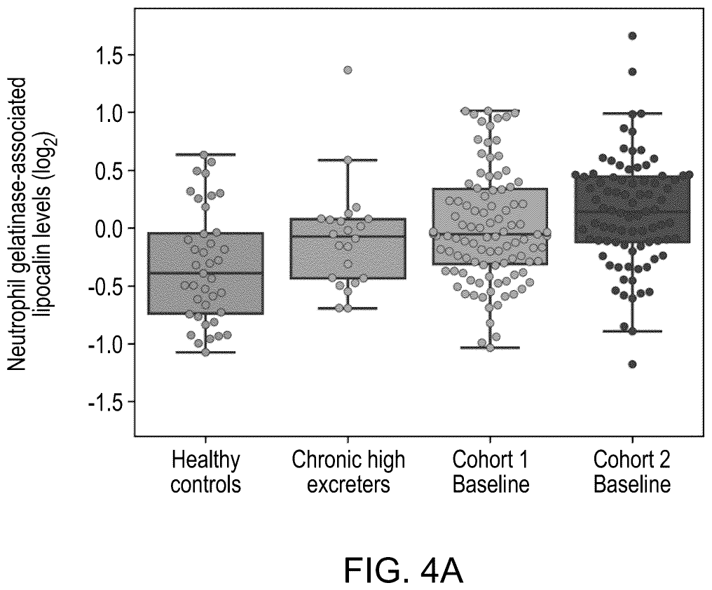

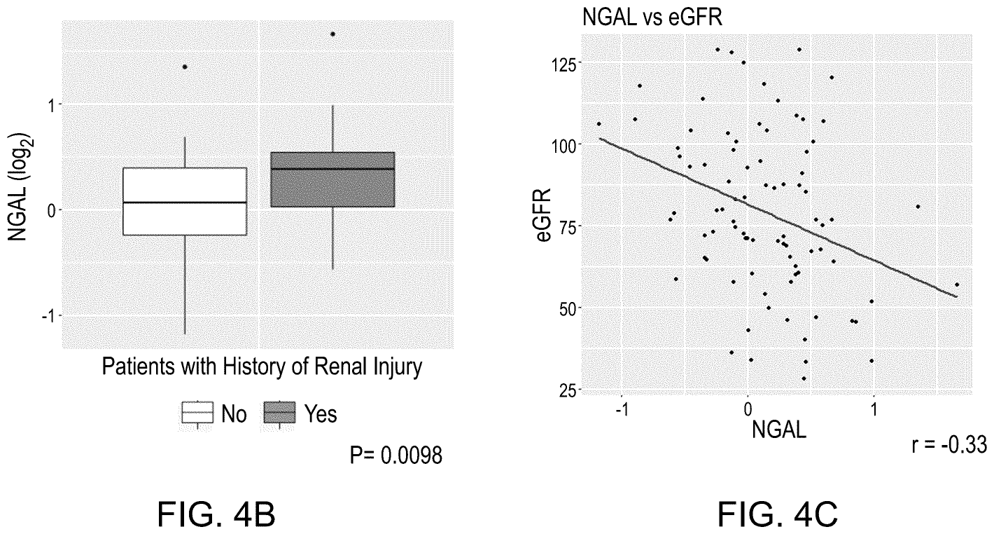

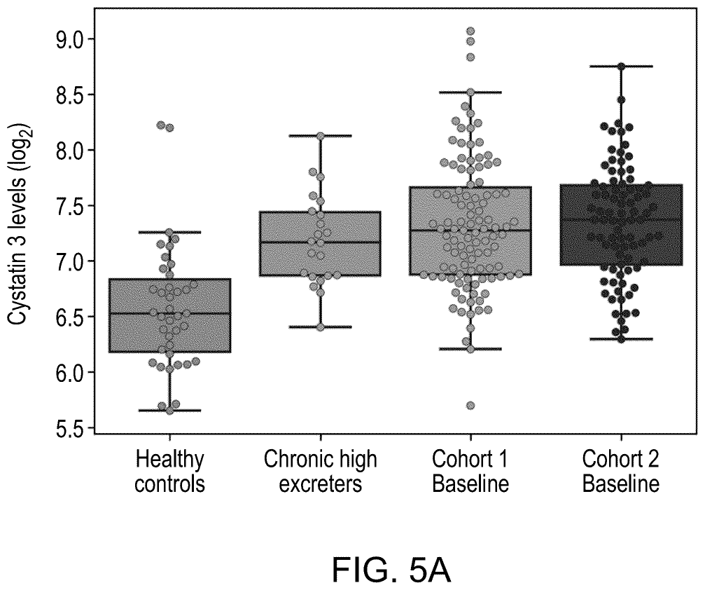

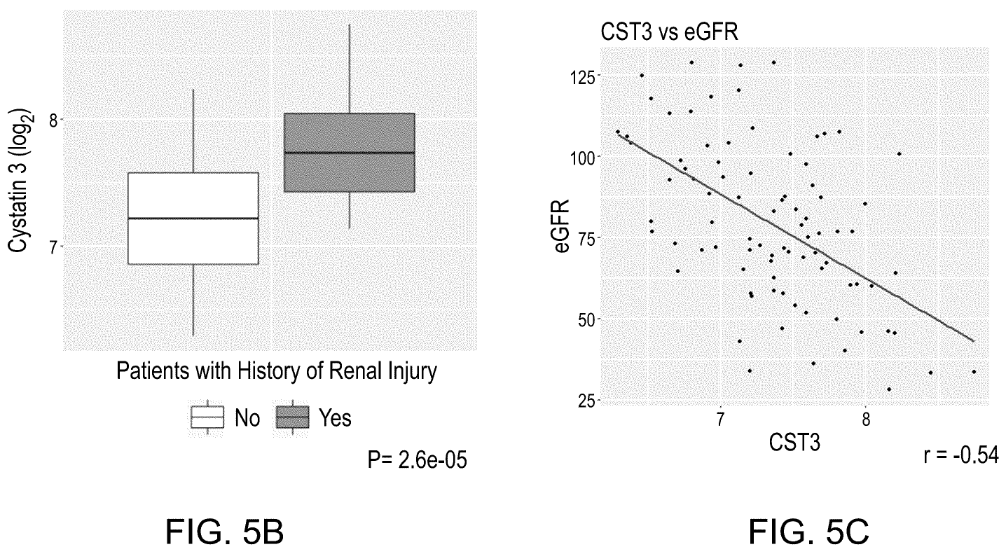

Additional renal injury biomarkers were selected to be explored based on measured OLINK® proteins from Example 1 and previous characterization of plasma levels and renal injury. Renal injury biomarkers neutrophil gelatinase-associated lipocalin (NGAL), cystatin 3 (CST3) and chitinase-3-like protein 1 (CHI3L1) showed significant elevations in patients with AHP (FIGS. 4A, 5A, and 6A, respectively). AHP patients with a diagnosis of renal disease demonstrated significantly higher levels of each of these biomarkers than patients without such a diagnosis (p-values<0.01) (FIGS. 4B, 5B, and 6B, respectively). Moderate to strong correlations were observed between each of these biomarkers and eGFR (correlation coefficients −0.33 to −0.54) (FIGS. 4C, 5C, and 6C, respectively). CST3 was also significantly elevated in CHE patients compared to controls (FIG. 5A).

As shown in the data presented in FIGS. 2A-2C, 3A-3C, 4A-4C, 5A-5C, and 6A-6C, biomarkers of renal injury, KIM1, MMP7, NGAL, CST3, and CHI3L1, were found to be elevated in AHP patients relative to healthy controls, KIM1, MMP7, and CST3 were also found to be elevated in CHE patients, and KIM1, MMP7, NGAL, CST3, and CHI3L1 were further elevated in patients with a previous diagnosis of renal injury and were moderately correlated with eGFR.

Accordingly, renal injury biomarkers may aid in diagnosing and managing kidney disease in patients with AHP suffering from recurrent acute attacks as well as chronic high excreters. These biomarkers may be useful for diagnosis and monitoring of renal injury in AHP patients.

EXEMPLARY SEQUENCES
KIM1

| | |
|---|---|
| LOCUS | NP_036338 364 aa linear PRI 26-SEP-2021 |
| DEFINITION | hepatitis A virus cellular receptor 1 isoform a precursor [Homo |
| | sapiens]. |
| ACCESSION | NP_036338 |
| VERSION | NP_036338.2 |

| | |
|---|---|
| 1 | mhpqvvilsl ilhladsvag svkvggeagp svtlpchysg avtsmcwnrg scslftcqng |
| 61 | ivwtngthvt yrkdtrykll gdlsrrdvsl tientavsds gvyccrvehr gwfndmkitv |
| 121 | sleivppkvt ttpivttvpt vttvrtsttv pttttvpmtt vptttvpttm sipttttvlt |
| 181 | tmtvstttsv ptttsipttt svpvtttvst fvppmplprq nhepvatsps spqpaethpt |
| 241 | tlqgairrep tssplysytt dgndtvtess dglwnnnqtq lflehsllta nttkgiyagv |
| 301 | cisvlvllal lgviiakkyf fkkevqqlsv sfsslqikal qnavekevqa edniyiensl |
| 361 | yatd (SEQ ID NO: 1) |

| | |
|---|---|
| LOCUS | NP_001166864 364 aa linear PRI 26-SEP-2021 |
| DEFINITION | hepatitis A virus cellular receptor 1 isoform a precursor [Homo |
| | sapiens]. |
| ACCESSION | NP_001166864 |
| VERSION | NP_001166864.1 |

| | |
|---|---|
| 1 | mhpqvvilsl ilhladsvag svkvggeagp svtlpchysg avtsmcwnrg scslftcqng |
| 61 | ivwtngthvt yrkdtrykll gdlsrrdvsl tientavsds gvyccrvehr gwfndmkitv |
| 121 | sleivppkvt ttpivttvpt vttvrtsttv pttttvpmtt vptttvpttm sipttttvlt |
| 181 | tmtvstttsv ptttsipttt svpvtttvst fvppmplprq nhepvatsps spqpaethpt |
| 241 | tlqgairrep tssplysytt dgndtvtess dglwnnnqtq lflehsllta nttkgiyagv |
| 301 | cisvlvllal lgviiakkyf fkkevqqlsv sfsslqikal qnavekevqa edniyiensl |
| 361 | yatd (SEQ ID NO: 2) |

| | |
|---|---|
| LOCUS | NP_001295085 401 aa linear PRI 26-SEP-2021 |
| DEFINITION | hepatitis A virus cellular receptor 1 isoform b precursor [Homo |
| | sapiens]. |
| ACCESSION | NP_001295085 XP_011532809 |
| VERSION | NP_001295085.1 |

| | |
|---|---|
| 1 | mhpqvvilsl ilhladsvag svkvggeagp svtlpchysg avtsmcwnrg scslftcqng |
| 61 | ivwtngthvt yrkdtrykll gdlsrrdvsl tientavsds gvyccrvehr gwfndmkitv |
| 121 | sleivppkvt ttpivttvpt vttvrtsttv pttttvpmtt vptttvpttm sipttttvlt |
| 181 | tmtvstttsv ptttsipttt svpvtttvst fvppmplprq nhepvatsps spqpaethpt |
| 241 | tlqgairrep tssplysytt dgndtvtess dglwnnnqtq lflehsllta nttkgiyagv |
| 301 | cisvlvllal lgviiakmfh laafklklck mqlkrkskqk tistlrivfm prtktqwcsl |
| 361 | rvyahecrrl nrhqhirrll dpktiflfqf hlafqhvsdt g (SEQ ID NO: 3) |

MMP7

| | |
|---|---|
| LOCUS | NP_002414 267 aa linear PRI 19-SEP-2021 |
| DEFINITION | matrilysin preproprotein [Homo sapiens]. |
| ACCESSION | NP_002414 |
| VERSION | NP_002414.1 |

| | |
|---|---|
| 1 | mrltvlcavc llpgslalpl pqeaggmsel qweqaqdylk rfylydsetk nansleaklk |
| 61 | emqkffglpi tgmlnsrvie imqkprcgvp dvaeyslfpn spkwtskvvt yrivsytrdl |
| 121 | phitvdrlvs kalnmwgkei plhfrkvvwg tadimigfar gahgdsypfd gpgntlahaf |
| 181 | apgtglggda hfdederwtd gsslginfly aathelghsl gmghssdpna vmyptygngd |
| 241 | pqnfklsqdd ikgiqklygk rsnsrkk (SEQ ID NO: 4) |

NGAL

| | |
|---|---|
| LOCUS | NP_005555 198 aa linear PRI 19-SEP-2021 |
| DEFINITION | neutrophil gelatinase-associated lipocalin precursor [Homo |
| | sapiens]. |
| ACCESSION | NP_005555 |
| VERSION | NP_005555.2 |

| | |
|---|---|
| 1 | mplgllwlgl allgalhaga qdstsdlipa pplskvplqq nfqdnqfqgk wyvvglagna |
| 61 | ilredkdpqk myatiyelke dksynvtsvl frkkkcdywi rtfvpgcqpg eftlgniksy |
| 121 | pgltsylvrv vstnynqham vffkkvsqnr eyfkitlygr tkeltselke nfirfskslg |
| 181 | lpenhivfpv pidqcidg (SEQ ID NO: 5) |

CST3

| | |
|---|---|
| LOCUS | NP_000090 146 aa linear PRI 12-SEP-2021 |
| DEFINITION | cystatin-C precursor [Homo sapiens]. |
| ACCESSION | NP_000090 |
| VERSION | NP_000090.1 |

| | |
|---|---|
| 1 | magplrapll llailavala vspaagsspg kpprlvggpm dasveeegvr raldfavgey |
| 61 | nkasndmyhs ralqvvrark qivagvnyfl dvelgrttct ktqpnldncp fhdqphlkrk |
| 121 | afcsfqiyav pwqgtmtlsk stcqda (SEQ ID NO: 6) |

| | |
|---|---|
| LOCUS | NP_001275543 146 aa linear PRI 12-SEP-2021 |
| DEFINITION | cystatin-C precursor [Homo sapiens]. |
| ACCESSION | NP_001275543 XP_005260729 |
| VERSION | NP_001275543.1 |

| | |
|---|---|
| 1 | magplrapll llailavala vspaagsspg kpprlvggpm dasveeegvr raldfavgey |
| 61 | nkasndmyhs ralqvvrark qivagvnyfl dvelgrttct ktqpnldncp fhdqphlkrk |
| 121 | afcsfqiyav pwqgtmtlsk stcqda (SEQ ID NO: 7) |

CHI3L1

| | |
|---|---|
| LOCUS | NP 001267 383 aa linear PRI 19-SEP-2021 |
| DEFINITION | chitinase-3-like protein 1 precursor [Homo sapiens]. |
| ACCESSION | NP_001267 |
| VERSION | NP_001267.2 |

| | |
|---|---|
| 1 | mgvkasqtgf vvlvllqccs ayklvcyyts wsqyregdgs cfpdaldrfl cthiiysfan |
| 61 | isndhidtwe wndvtlygml ntlknrnpnl ktllsvggwn fgsqrfskia sntqsrrtfi |
| 121 | ksvppflrth gfdgldlawl ypgrrdkqhf ttlikemkae fikeaqpgkk qlllsaalsa |
| 181 | gkvtidssyd iakisqhldf isimtydfhg awrgttghhs plfrgqedas pdrfsntdya |
| 241 | vgymlrlgap asklvmgipt fgrsftlass etgvgapisg pgipgrftke agtlayyeic |
| 301 | dflrgatvhr ilgqqvpyat kgnqwvgydd qesvkskvqy lkdrqlagam vwaldlddfq |
| 361 | gsfcgqdlrf pltnaikdal aat (SEQ ID NO: 8) |

APLP1

| | |
|---|---|
| LOCUS | NP_005157 650 aa linear PRI 02-JUL-2021 |
| DEFINITION | amyloid-like protein 1 isoform 2 precursor [Homo sapiens]. |
| ACCESSION | NP_005157 |
| VERSION | NP_005157.1 |

| | |
|---|---|
| 1 | mgpaspaarg lsrrpgqppl plllplllll Iraqpaigsl aggspgaaea pgsaqvaglc |
| 61 | grltlhrdlr tgrwepdpqr srrclrdpqr vleycrqmyp elqiarvega tqaipmerwc |
| 121 | ggsrsgscah phhqvvpfrc lpgefvseal lvpegcrflh qermdqcess trrhqeaqea |
| 181 | cssqglilhg sgmllpcgsd rfrgveyvcc pppgtpdpsg tavgdpstrs wppgsrvega |
| 241 | edeeeeesfp qpvddyfvep pqaeeeeetv pppsshtlav vgkvtptprp tdgvdiyfgm |
| 301 | pgeisehegf lrakmdleer rmrqinevmr ewamadnqsk nlpkadrqal nehfqsilqt |
| 361 | leeqvsgerq rlvethatrv ialindqrra alegflaalq adppqaervl lalrrylrae |
| 421 | qkeqrhtlrh yqhvaavdpe kaqqmrfqvh thlqvieerv nqslglldqn phlagelrpq |
| 481 | iqellhsehl gpseleapap ggssedkggl qppdskddtp mtlpkgsteq daaspekekm |
| 541 | npleqyerkv nasvprgfpf hsseiqrdel apagtgvsre avsgllimga gggslivlsm |
| 601 | lllrrkkpyg aishgvvevd pmltleeqql relqrhgyen ptyrfleerp (SEQ ID NO: 9) |

| | |
|---|---|
| LOCUS | NP_001019978 651 aa linear PRI 01-JUL-2021 |
| DEFINITION | amyloid-like protein 1 isoform 1 precursor [Homo sapiens]. |
| ACCESSION | NP_001019978 |
| VERSION | NP_001019978.1 |

| | |
|---|---|
| 1 | mgpaspaarg lsrrpgqppl plllplllll lraqpaigsl aggspgaaea pgsaqvaglc |
| 61 | grltlhrdlr tgrwepdpqr srrclrdpqr vleycrqmyp elqiarvega tqaipmerwc |
| 121 | ggsrsgscah phhqvvpfrc lpgefvseal lvpegerflh qermdqcess trrhqeaqea |
| 181 | cssqglilhg sgmllpcgsd rfrgveyvcc pppgtpdpsg tavgdpstrs wppgsrvega |
| 241 | edeeeeesfp qpvddyfvep pqaeeeeetv pppsshtlav vgkvtptprp tdgvdiyfgm |
| 301 | pgeisehegf lrakmdleer rmrqinevmr ewamadnqsk nlpkadrqal nehfqsilqt |
| 361 | leeqvsgerq rlvethatrv ialindqrra alegflaalq adppqaervl lalrrylrae |
| 421 | qkeqrhtlrh yqhvaavdpe kaqqmrfqvh thlqvieerv nqslglldqn phlagelrpq |
| 481 | iqellhsehl gpseleapap ggssedkggl qppdskdadt pmtlpkgste qdaaspekek |
| 541 | mnpleqyerk vnasvprgfp fhsseiqrde lapagtgvsr eavsgllimg agggslivls |
| 601 | mlllrrkkpy gaishgvvev dpmltleeqq lrelqrhgye nptyrfleer p (SEQ ID NO: 10) |

ALAS1

| | |
|---|---|
| LOCUS | NM_000688 2407 bp mRNA linear PRI 19-NOV-2011 |
| DEFINITION | Homo sapiens aminolevulinate, delta-, synthase 1 (ALAS1), |
| | transcript variant 1, mRNA. |
| ACCESSION | NM_000688 |
| VERSION | NM_000688.4 |

| | |
|---|---|
| 1 | ctgtatatta aggcgccggc gatcgcggcc tgaggctgct cccggacaag ggcaacgagc |
| 61 | gtttcgtttg gacttctcga cttgagtgcc cgcctccttc gccgccgcct ctgcagtcct |
| 121 | cagcgcagtt atgcccagtt cttcccgctg tggggacacg accacggagg aatccttgct |
| 181 | tcagggactc gggaccctgc tggacccctt cctcgggttt aggggatgtg gggaccagga |
| 241 | gaaagtcagg atccctaaga gtcttccctg cctggatgga tgagtggctt cttctccacc |
| 301 | tagattcttt ccacaggagc cagcatactt cctgaacatg gagagtgttg ttcgccgctg |
| 361 | cccattctta tcccgagtcc cccaggcctt tctgcagaaa gcaggcaaat ctctgttgtt |
| 421 | ctatgcccaa aactgcccca agatgatgga agttggggcc aagccagccc ctcgggcatt |
| 481 | gtccactgca gcagtacact accaacagat caaagaaacc cctccggcca gtgagaaaga |
| 541 | caaaactgct aaggccaagg tccaacagac tcctgatgga tcccagcaga gtccagatgg |
| 601 | cacacagctt ccgtctggac accccttgcc tgccacaagc cagggcactg caagcaaatg |
| 661 | ccctttcctg gcagcacaga tgaatcagag aggcagcagt gtcttctgca aagccagtct |
| 721 | tgagcttcag gaggatgtgc aggaaatgaa tgccgtgagg aaagaggttg ctgaaacctc |
| 781 | agcaggcccc agtgtggtta gtgtgaaaac cgatggaggg gatcccagtg gactgctgaa |
| 841 | gaacttccag gacatcatgc aaaagcaaag accagaaaga gtgtctcatc ttcttcaaga |
| 901 | taacttgcca aaatctgttt ccacttttca gtatgatcgt ttctttgaga aaaaaattga |
| 961 | tgagaaaaag aatgaccaca cctatcgagt ttttaaaact gtgaaccggc gagcacacat |
| 1021 | cttccccatg gcagatgact attcagactc cctcatcacc aaaaagcaag tgtcagtctg |
| 1081 | gtgcagtaat gactacctag gaatgagtcg ccacccacgg gtgtgtgggg cagttatgga |
| 1141 | cactttgaaa caacatggtg ctggggcagg tggtactaga aatatttctg gaactagtaa |
| 1201 | attccatgtg gacttagagc gggagctggc agacctccat gggaaagatg ccgcactctt |
| 1261 | gttttcctcg tgctttgtgg ccaatgactc aaccctcttc accctggcta agatgatgcc |
| 1321 | aggctgtgag atttactctg attctgggaa ccatgcctcc atgatccaag ggattcgaaa |
| 1381 | cagccgagtg ccaaagtaca tcttccgcca caatgatgtc agccacctca gagaactgct |
| 1441 | gcaaagatct gacccctcag tccccaagat tgtggcattt gaaactgtcc attcaatgga |
| 1501 | tggggcggtg tgcccactgg aagagctgtg tgatgtggcc catgagtttg gagcaatcac |
| 1561 | cttcgtggat gaggtccacg cagtggggct ttatggggct cgaggcggag ggattgggga |
| 1621 | tcgggatgga gtcatgccaa aaatggacat catttctgga acacttggca aagcctttgg |
| 1681 | ttgtgttgga gggtacatcg ccagcacgag ttctctgatt gacaccgtac ggtcctatgc |
| 1741 | tgctggcttc atcttcacca cctctctgcc acccatgctg ctggctggag ccctggagtc |
| 1801 | tgtgcggatc ctgaagagcg ctgagggacg ggtgcttcgc cgccagcacc agcgcaacgt |
| 1861 | caaactcatg agacagatgc taatggatgc cggcctccct gttgtccact gccccagcca |
| 1921 | catcatccct gtgcgggttg cagatgctgc taaaaacaca gaagtctgtg atgaactaat |
| 1981 | gagcagacat aacatctacg tgcaagcaat caattaccct acggtgcccc ggggagaaga |
| 2041 | gctcctacgg attgccccca cccctcacca cacaccccag atgatgaact acttccttga |
| 2101 | gaatctgcta gtcacatgga agcaagtggg gctggaactg aagcctcatt cctcagctga |
| 2161 | gtgcaacttc tgcaggaggc cactgcattt tgaagtgatg agtgaaagag agaagtccta |
| 2221 | tttctcaggc ttgagcaagt tggtatctgc tcaggcctga gcatgacctc aattatttca |
| 2281 | cttaacccca ggccattatc atatccagat ggtcttcaga gttgtcttta tatgtgaatt |
| 2341 | aagttatatt aaattttaat ctatagtaaa aacatagtcc tggaaataaa ttcttgctta |
| 2401 | aatggtg (SEQ ID NO: 11) |

| | |
|---|---|
| LOCUS | NM_199166 2258 bp mRNA linear PRI 19-NOV-2011 |
| DEFINITION | Homo sapiens aminolevulinate, delta-, synthase 1 (ALAS1), |
| | transcript variant 2, mRNA. |
| ACCESSION | NM_199166 |
| VERSION | NM_199166.1 |

| | |
|---|---|
| 1 | caaggcgcat gcgcagcggt cactcccgct gtatattaag gcgccggcga tcgcggcctg |
| 61 | aggctgctcc cggacaaggg caacgagcgt ttcgtttgga cttctcgact tgagtgcccg |
| 121 | cctccttcgc cgccgcctct gcagtcctca gcgcagtctt tccacaggag ccagcatact |
| 181 | tcctgaacat ggagagtgtt gttcgccgct gcccattctt atcccgagtc ccccaggcct |
| 241 | ttctgcagaa agcaggcaaa tctctgttgt tctatgccca aaactgcccc aagatgatgg |
| 301 | aagttggggc caagccagcc cctcgggcat tgtccactgc agcagtacac taccaacaga |
| 361 | tcaaagaaac ccctccggcc agtgagaaag acaaaactgc taaggccaag gtccaacaga |
| 421 | ctcctgatgg atcccagcag agtccagatg gcacacagct tccgtctgga caccccttgc |
| 481 | ctgccacaag ccagggcact gcaagcaaat gccctttcct ggcagcacag atgaatcaga |
| 541 | gaggcagcag tgtcttctgc aaagccagtc ttgagcttca ggaggatgtg caggaaatga |
| 601 | atgccgtgag gaaagaggtt gctgaaacct cagcaggccc cagtgtggtt agtgtgaaaa |
| 661 | ccgatggagg ggatcccagt ggactgctga agaacttcca ggacatcatg caaaagcaaa |
| 721 | gaccagaaag agtgtctcat cttcttcaag ataacttgcc aaaatctgtt tccacttttc |
| 781 | agtatgatcg tttctttgag aaaaaaattg atgagaaaaa gaatgaccac acctatcgag |
| 841 | tttttaaaac tgtgaaccgg cgagcacaca tcttccccat ggcagatgac tattcagact |
| 901 | ccctcatcac caaaaagcaa gtgtcagtct ggtgcagtaa tgactaccta ggaatgagtc |
| 961 | gccacccacg ggtgtgtggg gcagttatgg acactttgaa acaacatggt gctggggcag |
| 1021 | gtggtactag aaatatttct ggaactagta aattccatgt ggacttagag cgggagctgg |
| 1081 | cagacctcca tgggaaagat gccgcactct tgttttcctc gtgctttgtg gccaatgact |
| 1141 | caaccctctt caccctggct aagatgatgc caggctgtga gatttactct gattctggga |
| 1201 | accatgcctc catgatccaa gggattcgaa acagccgagt gccaaagtac atcttccgcc |
| 1261 | acaatgatgt cagccacctc agagaactgc tgcaaagatc tgacccctca gtccccaaga |
| 1321 | ttgtggcatt tgaaactgtc cattcaatgg atggggcggt gtgcccactg gaagagctgt |
| 1381 | gtgatgtggc ccatgagttt ggagcaatca ccttcgtgga tgaggtccac gcagtggggc |
| 1441 | tttatggggc tcgaggcgga gggattgggg atcgggatgg agtcatgcca aaaatggaca |
| 1501 | tcatttctgg aacacttggc aaagcctttg gttgtgttgg agggtacatc gccagcacga |
| 1561 | gttctctgat tgacaccgta cggtcctatg ctgctggctt catcttcacc acctctctgc |
| 1621 | cacccatgct gctggctgga gccctggagt ctgtgcggat cctgaagagc gctgagggac |
| 1681 | gggtgcttcg ccgccagcac cagcgcaacg tcaaactcat gagacagatg ctaatggatg |
| 1741 | ccggcctccc tgttgtccac tgccccagcc acatcatccc tgtgcgggtt gcagatgctg |
| 1801 | ctaaaaacac agaagtctgt gatgaactaa tgagcagaca taacatctac gtgcaagcaa |
| 1861 | tcaattaccc tacggtgccc cggggagaag agctcctacg gattgccccc acccctcacc |
| 1921 | acacacccca gatgatgaac tacttccttg agaatctgct agtcacatgg aagcaagtgg |
| 1981 | ggctggaact gaagcctcat tcctcagctg agtgcaactt ctgcaggagg ccactgcatt |
| 2041 | ttgaagtgat gagtgaaaga gagaagtcct atttctcagg cttgagcaag ttggtatctg |
| 2101 | ctcaggcctg agcatgacct caattatttc acttaacccc aggccattat catatccaga |
| 2161 | tggtcttcag agttgtcttt atatgtgaat taagttatat taaattttaa tctatagtaa |
| 2221 | aaacatagtc ctggaaataa attcttgctt aaatggtg (SEQ ID NO: 12) |

1. A method of treating a human subject having acute hepatic porphyria (AHP), comprising:
responsive to a determination of an elevated level of a biomarker in a subject relative to a reference level, administering to the subject a therapeutic agent that reduces expression of 5′-aminolevulinic acid synthase 1 (ALAS1),
thereby treating the subject.

2. A method of treating a human subject having, or at risk of having, AHP, comprising:
obtaining or having obtained a biological sample from the subject,
performing or having performed an assay to determine the level of a biomarker in the biological sample,
and, if the subject has an elevated level of the biomarker relative to a reference value, administering to the subject a therapeutic agent that reduces expression of ALAS1,
thereby treating the subject.

3. (canceled)

4. The method of claim 2, wherein the biomarker is selected from the group consisting of KIM1, APLP1, MMP7, NGAL, CST3, and CHI3L1.

5. The method of claim 2, wherein the biomarker is a renal injury biomarker.

6. (canceled)

7. The method of claim 2, wherein the therapeutic agent that reduces the expression of ALAS1 is a nucleic acid therapeutic.

8. The method of claim 7, wherein the nucleic acid therapeutic is an RNAi agent or an antisense oligonucleotide.

9. The method of claim 7, wherein the nucleic acid therapeutic is givosiran.

10. The method of claim 2, wherein the subject:
(i) is a chronic high excreter;
(ii) has recurring acute attacks;
(iii) has an elevated level of ALA and/or PBG;
(iv) has a mutation associated with AHP;
(v) has a history of renal impairment;
(vi) has been diagnosed with a renal injury; and/or
(vii) has a reduced estimated glomerular filtration rate (eGFR).

11. (canceled)

12. The method of claim 2, wherein the subject:
(i) has not been diagnosed with AHP;
(ii) has not been diagnosed as having a porphyria;
(iii) does not meet the diagnostic criteria for AHP; and/or
(iv) does not have a mutation associated with AHP.

13.-20. (canceled)

21. The method of claim 2, wherein the reference level is a healthy control level or an earlier level in the same subject.

22. The method of claim 2, wherein the level of the biomarker is increased by at least 2-fold, compared to the reference level of the biomarker.

23. The method of claim 2, wherein the subject is being treated with a second therapeutic agent.

24. The method of claim 23, wherein the second therapeutic agent comprises a heme product, glucose, dextrose, or combinations thereof.

25. The method of claim 23, further comprising discontinuation of treatment with the second therapeutic agent when the subject has the elevated level of the biomarker.

26. The method of claim 2, wherein the level is determined in a subject sample selected from blood, plasma, serum, urine, or stool.

27. The method of claim 2, wherein the subject is further suffering from one or more symptoms associated with AHP.

28.-53. (canceled)

54. An in vitro method of diagnosing AHP in a subject, the method comprising:
(a) determining the level of a biomarker in a sample from the subject;
(b) comparing the level of the biomarker determined in step (a) to a reference level of the biomarker, e.g., renal injury biomarker; and
(c) assessing whether the subject suffers from AHP, wherein an increase in the level of the biomarker determined in step (a) as compared to the reference level of the biomarker, e.g., renal injury biomarker, is indicative of the subject suffering from AHP.

55. The method of claim 54, wherein the biomarker is chosen from one or more of KIM1, APLP1, MMP7, NGAL, CST3, or CHI3L1.

56. The method of claim 54, wherein the biomarker is a renal injury biomarker.

57. The method of claim 56, wherein the renal injury biomarker is chosen from one or more (e.g., 2, 3, 4, or all) of KIM1, APLP1, MMP7, NGAL, CST3, or CHI3L1.

58. The method of claim 54, wherein the subject:
(i) has been diagnosed with AHP;
(ii) has an elevated level of ALA and/or PBG;
(iii) has been diagnosed with a renal injury; or
(iv) has a reduced estimated glomerular filtration rate (eGFR).

59. (canceled)

60. The method of claim 54, wherein the subject has a mutation associated with AHP, or wherein the subject does not have a mutation associated with AHP.

61.-63. (canceled)

64. The method of claim 54, wherein the reference level is a healthy control level or an earlier level in the same subject.

65. The method of claim 54, wherein the level of the biomarker is increased by at least 2-fold, compared to the reference level of the biomarker.

66. The method of claim 54, wherein the subject is being treated with a therapeutic agent that reduces the expression of ALAS1.

67.-91. (canceled)


SEQUENCE LISTING (19 sequences)

SEQ ID NO: 1 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 2 - protein, 364 aa, Homo sapiens; identical to SEQ ID NO: 1

SEQ ID NO: 3 - protein, 401 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKMFHLAAFKLKLCKMQLKRKSKQKTISTLRIVFMPRTKTQWCSLRVYAHECRRLNRHQHIRRLLDPKTIFLFQFHLAFQHVSDTG

SEQ ID NO: 4 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 5 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 6 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 7 - protein, 146 aa, Homo sapiens; identical to SEQ ID NO: 6

SEQ ID NO: 8 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 9 - protein, 650 aa, Homo sapiens
MGPASPAARGLSRRPGQPPLPLLLPLLLLLLRAQPAIGSLAGGSPGAAEAPGSAQVAGLCGRLTLHRDLRTGRWEPDPQRSRRCLRDPQRVLEYCRQMYPELQIARVEQATQAIPMERWCGGSRSGSCAHPHHQVVPFRCLPGEFVSEALLVPEGCRFLHQERMDQCESSTRRHQEAQEACSSQGLILHGSGMLLPCGSDRFRGVEYVCCPPPGTPDPSGTAVGDPSTRSWPPGSRVEGAEDEEEEESFPQPVDDYFVEPPQAEEEEETVPPPSSHTLAVVGKVTPTPRPTDGVDIYFGMPGEISEHEGFLRAKMDLEERRMRQINEVMREWAMADNQSKNLPKADRQALNEHFQSILQTLEEQVSGERQRLVETHATRVIALINDQRRAALEGFLAALQADPPQAERVLLALRRYLRAEQKEQRHTLRHYQHVAAVDPEKAQQMRFQVHTHLQVIEERVNQSLGLLDQNPHLAQELRPQIQELLHSEHLGPSELEAPAPGGSSEDKGGLQPPDSKDDTPMTLPKGSTEQDAASPEKEKMNPLEQYERKVNASVPRGFPFHSSEIQRDELAPAGTGVSREAVSGLLIMGAGGGSLIVLSMLLLRRKKPYGAISHGVVEVDPMLTLEEQQLRELQRHGYENPTYRFLEERP

SEQ ID NO: 10 - protein, 651 aa, Homo sapiens
MGPASPAARGLSRRPGQPPLPLLLPLLLLLLRAQPAIGSLAGGSPGAAEAPGSAQVAGLCGRLTLHRDLRTGRWEPDPQRSRRCLRDPQRVLEYCRQMYPELQIARVEQATQAIPMERWCGGSRSGSCAHPHHQVVPFRCLPGEFVSEALLVPEGCRFLHQERMDQCESSTRRHQEAQEACSSQGLILHGSGMLLPCGSDRFRGVEYVCCPPPGTPDPSGTAVGDPSTRSWPPGSRVEGAEDEEEEESFPQPVDDYFVEPPQAEEEEETVPPPSSHTLAVVGKVTPTPRPTDGVDIYFGMPGEISEHEGFLRAKMDLEERRMRQINEVMREWAMADNQSKNLPKADRQALNEHFQSILQTLEEQVSGERQRLVETHATRVIALINDQRRAALEGFLAALQADPPQAERVLLALRRYLRAEQKEQRHTLRHYQHVAAVDPEKAQQMRFQVHTHLQVIEERVNQSLGLLDQNPHLAQELRPQIQELLHSEHLGPSELEAPAPGGSSEDKGGLQPPDSKDADTPMTLPKGSTEQDAASPEKEKMNPLEQYERKVNASVPRGFPFHSSEIQRDELAPAGTGVSREAVSGLLIMGAGGGSLIVLSMLLLRRKKPYGAISHGVVEVDPMLTLEEQQLRELQRHGYENPTYRFLEERP

SEQ ID NO: 11 - DNA, 2407 bp, Homo sapiens
  source 1..2407: mol_type = genomic DNA
ctgtatattaaggcgccggcgatcgcggcctgaggctgctcccggacaagggcaacgagcgtttcgtttggacttctcgacttgagtgcccgcctccttcgccgccgcctctgcagtcctcagcgcagttatgcccagttcttcccgctgtggggacacgaccacggaggaatccttgcttcagggactcgggaccctgctggaccccttcctcgggtttaggggatgtggggaccaggagaaagtcaggatccctaagagtcttccctgcctggatggatgagtggcttcttctccacctagattctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacatcatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtg

SEQ ID NO: 12 - DNA, 2258 bp, Homo sapiens
  source 1..2258: mol_type = genomic DNA
caaggcgcatgcgcagcggtcactcccgctgtatattaaggcgccggcgatcgcggcctgaggctgctcccggacaagggcaacgagcgtttcgtttggacttctcgacttgagtgcccgcctccttcgccgccgcctctgcagtcctcagcgcagtctttccacaggagccagcatacttcctgaacatggagagtgttgttcgccgctgcccattcttatcccgagtcccccaggcctttctgcagaaagcaggcaaatctctgttgttctatgcccaaaactgccccaagatgatggaagttggggccaagccagcccctcgggcattgtccactgcagcagtacactaccaacagatcaaagaaacccctccggccagtgagaaagacaaaactgctaaggccaaggtccaacagactcctgatggatcccagcagagtccagatggcacacagcttccgtctggacaccccttgcctgccacaagccagggcactgcaagcaaatgccctttcctggcagcacagatgaatcagagaggcagcagtgtcttctgcaaagccagtcttgagcttcaggaggatgtgcaggaaatgaatgccgtgaggaaagaggttgctgaaacctcagcaggccccagtgtggttagtgtgaaaaccgatggaggggatcccagtggactgctgaagaacttccaggacatcatgcaaaagcaaagaccagaaagagtgtctcatcttcttcaagataacttgccaaaatctgtttccacttttcagtatgatcgtttctttgagaaaaaaattgatgagaaaaagaatgaccacacctatcgagtttttaaaactgtgaaccggcgagcacacatcttccccatggcagatgactattcagactccctcatcaccaaaaagcaagtgtcagtctggtgcagtaatgactacctaggaatgagtcgccacccacgggtgtgtggggcagttatggacactttgaaacaacatggtgctggggcaggtggtactagaaatatttctggaactagtaaattccatgtggacttagagcgggagctggcagacctccatgggaaagatgccgcactcttgttttcctcgtgctttgtggccaatgactcaaccctcttcaccctggctaagatgatgccaggctgtgagatttactctgattctgggaaccatgcctccatgatccaagggattcgaaacagccgagtgccaaagtacatcttccgccacaatgatgtcagccacctcagagaactgctgcaaagatctgacccctcagtccccaagattgtggcatttgaaactgtccattcaatggatggggcggtgtgcccactggaagagctgtgtgatgtggcccatgagtttggagcaatcaccttcgtggatgaggtccacgcagtggggctttatggggctcgaggcggagggattggggatcgggatggagtcatgccaaaaatggacatcatttctggaacacttggcaaagcctttggttgtgttggagggtacatcgccagcacgagttctctgattgacaccgtacggtcctatgctgctggcttcatcttcaccacctctctgccacccatgctgctggctggagccctggagtctgtgcggatcctgaagagcgctgagggacgggtgcttcgccgccagcaccagcgcaacgtcaaactcatgagacagatgctaatggatgccggcctccctgttgtccactgccccagccacatcatccctgtgcgggttgcagatgctgctaaaaacacagaagtctgtgatgaactaatgagcagacataacatctacgtgcaagcaatcaattaccctacggtgccccggggagaagagctcctacggattgcccccacccctcaccacacaccccagatgatgaactacttccttgagaatctgctagtcacatggaagcaagtggggctggaactgaagcctcattcctcagctgagtgcaacttctgcaggaggccactgcattttgaagtgatgagtgaaagagagaagtcctatttctcaggcttgagcaagttggtatctgctcaggcctgagcatgacctcaattatttcacttaaccccaggccattatcatatccagatggtcttcagagttgtctttatatgtgaattaagttatattaaattttaatctatagtaaaaacatagtcctggaaataaattcttgcttaaatggtg

SEQ ID NO: 13 - RNA, 23 nt, synthetic construct
  source 1..23: mol_type = other RNA
taagatgagacactctttctggt

SEQ ID NO: 14 - RNA, 23 nt, synthetic construct
  source 1..23: mol_type = other RNA
  modified_base 16: mod_base = OTHER; note = Thymine
taagatgagacactctttctggt

SEQ ID NO: 15 - RNA, 21 nt, synthetic construct
  source 1..21: mol_type = other RNA
cagaaagagtgtctcatctta

SEQ ID NO: 16 - RNA, 21 nt, synthetic construct
  source 1..21: mol_type = other RNA
  modified_base 1: mod_base = OTHER; note = 2'-O-methylcytidine-3'-phosphate phosphorothioate
  modified_base 2: mod_base = OTHER; note = 2'-O-methyladenosine-3'-phosphate phosphorothioate
  modified_base 3: mod_base = OTHER; note = 2'-O-methylguanosine-3'-phosphate
  modified_base 4: mod_base = OTHER; note = 2'-O-methyladenosine-3'-phosphate
  modified_base 5: mod_base = OTHER; note = 2'-O-methyladenosine-3'-phosphate
  modified_base 6: mod_base = OTHER; note = 2'-O-methyladenosine-3'-phosphate
  modified_base 7: mod_base = OTHER; note = 2'-fluoroguanosine-3'-phosphate
  modified_base 8: mod_base = OTHER; note = 2'-O-methyladenosine-3'-phosphate
  modified_base 9: mod_base = OTHER; note = 2'-fluoroguanosine-3'-phosphate
  modified_base 10: mod_base = OTHER; note = 2'-O-methyluridine-3'-phosphate
  modified_base 11: mod_base = OTHER; note = 2'-fluoroguanosine-3'-phosphate
  modified_base 12: mod_base = OTHER; note = 2'-O-methyluridine-3'-phosphate
  modified_base 13: mod_base = OTHER; note = 2'-fluorocytidine-3'-phosphate
  modified_base 14: mod_base = OTHER; note = 2'-O-methyluridine-3'-phosphate
  modified_base 15: mod_base = OTHER; note = 2'-fluorocytidine-3'-phosphate
  modified_base 16: mod_base = OTHER; note = 2'-O-methyladenosine-3'-phosphate
  modified_base 17: mod_base = OTHER; note = 2'-O-methyluridine-3'-phosphate
  modified_base 18: mod_base = OTHER; note = 2'-O-methylcytidine-3'-phosphate
  modified_base 19: mod_base = OTHER; note = 2'-O-methyluridine-3'-phosphate
  modified_base 20: mod_base = OTHER; note = 2'-O-methyluridine-3'-phosphate
  modified_base 21: mod_base = OTHER; note = 2'-O-methyladenosine-3'-phosphate N-[tris(GalNAc-alkyl)-amidodecanoyl)]-4-hydroxyprolinol
cagaaagagtgtctcatctta

SEQ ID NO: 17 - RNA, 23 nt, synthetic construct
  source 1..23: mol_type = other RNA
  modified_base 1: mod_base = OTHER; note = 2'-O-methyluridine-3'-phosphate phosphorothioate
  modified_base 2: mod_base = OTHER; note = 2'-fluoroadenosine-3'-phosphate phosphorothioate
  modified_base 3: mod_base = OTHER; note = 2'-fluoroadenosine-3'-phosphate
  modified_base 4: mod_base = OTHER; note = 2'-fluoroguanosine-3'-phosphate
  modified_base 5: mod_base = OTHER; note = 2'-O-methyladenosine-3'-phosphate
  modified_base 6: mod_base = OTHER; note = 2'-fluorouridine-3'-phosphate
  modified_base 7: mod_base = OTHER; note = 2'-O-methylguanosine-3'-phosphate
  modified_base 8: mod_base = OTHER; note = 2'-fluoroadenosine-3'-phosphate
  modified_base 9: mod_base = OTHER; note = 2'-O-methylguanosine-3'-phosphate
  modified_base 10: mod_base = OTHER; note = 2'-fluoroadenosine-3'-phosphate
  modified_base 11: mod_base = OTHER; note = 2'-O-methylcytidine-3'-phosphate
  modified_base 12: mod_base = OTHER; note = 2'-fluoroadenosine-3'-phosphate
  modified_base 13: mod_base = OTHER; note = 2'-O-methylcytidine-3'-phosphate
  modified_base 14: mod_base = OTHER; note = 2'-fluorouridine-3'-phosphate
  modified_base 15: mod_base = OTHER; note = 2'-O-methylcytidine-3'-phosphate
  modified_base 16: mod_base = OTHER; note = 2'-fluorouridine-3'-phosphate
  modified_base 17: mod_base = OTHER; note = 2'-O-methyluridine-3'-phosphate
  modified_base 18: mod_base = OTHER; note = 2'-fluorouridine-3'-phosphate
  modified_base 19: mod_base = OTHER; note = 2'-O-methylcytidine-3'-phosphate
  modified_base 20: mod_base = OTHER; note = 2'-fluorouridine-3'-phosphate
  modified_base 21: mod_base = OTHER; note = 2'-O-methylguanosine-3'-phosphate phosphorothioate
  modified_base 22: mod_base = OTHER; note = 2'-O-methylguanosine-3'-phosphate phosphorothioate
  modified_base 23: mod_base = OTHER; note = 2'-O-methyluridine-3'-phosphate
taagatgagacactctttctggt

SEQ ID NO: 18 - RNA, 21 nt, synthetic construct
  source 1..21: mol_type = other RNA
  modified_base 1: mod_base = OTHER; note = Hydroxyl 2'-O-methylcytidine phosphorothioate
  modified_base 2: mod_base = OTHER; note = 2'-O-methyladenosine phosphorothioate
  modified_base 3: mod_base = OTHER; note = 2'-O-methylguanosine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-fluoroguanosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-O-methyluridine
  modified_base 11: mod_base = OTHER; note = 2'-fluoroguanosine
  modified_base 12: mod_base = OTHER; note = 2'-O-methyluridine
  modified_base 13: mod_base = OTHER; note = 2'-fluorocytidine
  modified_base 14: mod_base = OTHER; note = 2'-O-methyluridine
  modified_base 15: mod_base = OTHER; note = 2'-fluorocytidine
  modified_base 16: mod_base = OTHER; note = 2'-O-methyladenosine
  modified_base 17: mod_base = OTHER; note = 2'-O-methyluridine
  modified_base 18: mod_base = OTHER; note = 2'-O-methylcytidine
  modified_base 19: mod_base = OTHER; note = 2'-O-methyluridine
  modified_base 20: mod_base = OTHER; note = 2'-O-methyluridine
  modified_base 21: mod_base = OTHER; note = 2'-O-methyladenosine-N-[tris(GalNAc-alkyl)-amidodec anoyl)]-4-hydroxyprolinol
cagaaagagtgtctcatctta

SEQ ID NO: 19 - RNA, 23 nt, synthetic construct
  source 1..23: mol_type = other RNA
  modified_base 1: mod_base = OTHER; note = Hydroxyl 2'-O-methyluridine phosphorothioate
  modified_base 2: mod_base = OTHER; note = 2'-fluoroadenosine phosphorothioate
  modified_base 3: mod_base = OTHER; note = 2'-fluoroadenosine
  modified_base 4: mod_base = OTHER; note = 2'-fluoroguanosine
  modified_base 5: mod_base = OTHER; note = 2'-O-methyladenosine
  modified_base 6: mod_base = OTHER; note = 2'-fluorouridine
  modified_base 7: mod_base = OTHER; note = 2'-O-methylguanosine
  modified_base 8: mod_base = OTHER; note = 2'-fluoroadenosine
  modified_base 9: mod_base = OTHER; note = 2'-O-methylguanosine
  modified_base 10: mod_base = OTHER; note = 2'-fluoroadenosine
  modified_base 11: mod_base = OTHER; note = 2'-O-methylcytidine
  modified_base 12: mod_base = OTHER; note = 2'-fluoroadenosine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluorouridine
  modified_base 17: mod_base = OTHER; note = 2'-O-methyluridine
  modified_base 18: mod_base = OTHER; note = 2'-fluorouridine
  modified_base 19: mod_base = OTHER; note = 2'-O-methylcytidine
  modified_base 20: mod_base = OTHER; note = 2'-fluorouridine
  modified_base 21: mod_base = OTHER; note = 2'-O-methylguanosine phosphorothioate
  modified_base 22: mod_base = OTHER; note = 2'-O-methylguanosine phosphorothioate
  modified_base 23: mod_base = OTHER; note = 2'-O-methyluridine hydroxyl
taagatgagacactctttctggt